SEQUENCE LISTING (57 sequences)

SEQ ID NO: 1 - DNA, 6936 bp, Homo sapiens
aggccgcgctcagcaggcggggcgggagccgcgtgcgcccgaggacccggccggaaggcttgcgccagctcaggatgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcaactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataagtctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaatataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctgggccctgattacacagcaatagctatcatagttgccacactaagtatcttagttctcatgggcggactgatttggttcctcttccaaaggcaccgtttgcacctggcgggtttctcatcagttcgatatgcacaaggagtgaatgaagatgagattatgcttccttctttccatgactaaattcttctaaaagttttctaatttgcactaatgtgttatgagaaattagtcacttaaaatgtcccagtgtcagtatttactctgctccaaagtagaactcttaaatactttttcagttgtttagatcttaggcatgtgctggtatccacagttaattccctgctaaatgccatgtttatcaccctaattaatagaatggaggggactccaaagctggaactgaagtccaaattgtttgtacagtaatatgtttaatgttcattttctctgtatgaatgtgattggtaactaggatatgtatattttaatagaatttttaacaaaacttcttagaaaattaaaataggcatattactaggtgacatgtctactttttaatttttaagagcatccggccaaatgcaaaattagtacctcaaagtaaaaattgaactgtaaactctatcagcattgtttcaaaatagtcatttttagcactggggaaaaataaacaataagacatgcttactttttaatttttatttttttgagactgagtctctctctgttgcccaggctggagtacaatggcgtgatctcggctcactgcaaatctccgcctcccaggttcaagcgattctcctgcctcagcctcctgagtagctgggattacaggcaactgccaccatgcccggctaatttttgtatttttagtagagatggggtttcaccatgttggccaggctggtctcgaactcgtgaccgcaggtgatcctcccgcctcggcctcccaaagtgctgggattacaggcatgagccaccgcgcctggcctctgcttactttttatatagcaaaatgattcctcttggcaagatgtttcttatattattccaaagttatttcataccattattatgtaaatatgaagagtttttttctgtttataattgtttataaaacaatgacttttaaagatttagtgcttaacattttcccaagtgtgggaacattatttttagattgagtaggtaccttgtagcagtgtgctttgcattttctgatgtattacatgactgtttcttttgtaaagagaatcaactaggtatttaagactgataattttacaatttatatgcttcacatagcatgtcaacttttgactaagaattttgttttacttttttaacatgtgttaaacagagaaagggtccatgaaggaaagtgtatgagttgcatttgtaaaaatgagactttttcagtggaactctaaaccttgtgatgactactaacaaatgtaaaattatgagtgattaagaaaacattgctttgtggttatcactttaagttttgacacctagattatagtcttagtaatagcatccactggaaaaggtgaaaatgttttattcggcatttaacttacatttgtactttatttttgtataaaatccatagatttattttacatttagagtatttacactatgataaagttgtaaataattttctaagacagtttttatatagtctacagttgtcctgatttcttattgaatttgttagactagttctcttgtcctgtgatctgtgtacaattttagtcactaagactttcctccaagaactaagccaacttgatgtgaaaagcacagctgtatataatggtgatgtcataataaagttgttttatcttttaagtaaaagtaaaaaaaa

SEQ ID NO: 2 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 3 - DNA, 1616 bp, Homo sapiens
gccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 4 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 5 - protein, 21 aa, Artificial Sequence
  T-helper epitope
FNNFTVSFWLRVPKVSASHLE

SEQ ID NO: 6 - protein, 16 aa, Artificial Sequence
  T-helper cell epitope
GALNNRFQIKGVELKS

SEQ ID NO: 7 - protein, 15 aa, Artificial Sequence
  t-helper cell epitope
ALNNRFQIKGVELKS

SEQ ID NO: 8 - protein, 15 aa, Artificial Sequence
  t-helper cell epitope
LSEIKGVIVHRLEGV

SEQ ID NO: 9 - protein, 17 aa, Artificial Sequence
  t-helper cell epitope
TAAQITAGIALHQSNLN

SEQ ID NO: 10 - protein, 17 aa, Artificial Sequence
  t-helper cell epitope
IGTDNVHYKIMTRPSHQ

SEQ ID NO: 11 - protein, 17 aa, Artificial Sequence
  t-helper cell epitope
YKIMTRPSHQYLVIKLI

SEQ ID NO: 12 - protein, 17 aa, Artificial Sequence
  t-helper cell epitope
SHQYLVIKLIPNASLIE

SEQ ID NO: 13 - protein, 17 aa, Artificial Sequence
  t-helper cell epitope
KLIPNASLIENCTKAEL

SEQ ID NO: 14 - protein, 17 aa, Artificial Sequence
  t-helper cell epitope
LIENCTKAELGEYEKLL

SEQ ID NO: 15 - protein, 17 aa, Artificial Sequence
  t-helper cell epitope
AELGEYEKLLNSVLEPI

SEQ ID NO: 16 - protein, 17 aa, Artificial Sequence
  t-helper cell epitope
KLLNSVLEPINQALTLM

SEQ ID NO: 17 - protein, 17 aa, Artificial Sequence
  t-helper cell epitope
EPINQALTLMTKNVKPL

SEQ ID NO: 18 - protein, 17 aa, Artificial Sequence
  t-helper cell epitope
TLMTKNVKPLQSLGSGR

SEQ ID NO: 19 - protein, 17 aa, Artificial Sequence
  t-helper cell epitope
KPLQSLGSGRRQRRFAG

SEQ ID NO: 20 - protein, 17 aa, Artificial Sequence
  t-helper cell epitope
SGRRQRRFAGVVLAGVA

SEQ ID NO: 21 - protein, 17 aa, Artificial Sequence
  t-helper cell epitope
FAGVVLAGVALGVATAA

SEQ ID NO: 22 - protein, 17 aa, Artificial Sequence
  t-helper cell epitope
GVALGVATAAQITAGIA

SEQ ID NO: 23 - protein, 17 aa, Artificial Sequence
  t-helper cell epitope
GIALHQSNLNAQAIQSL

SEQ ID NO: 24 - protein, 17 aa, Artificial Sequence
  t-helper cell epitope
NLNAQAIQSLRTSLEQS

SEQ ID NO: 25 - protein, 17 aa, Artificial Sequence
  t-helper cell epitope
QSLRTSLEQSNKAIEEI

SEQ ID NO: 26 - protein, 17 aa, Artificial Sequence
  t-helper cell epitope
EQSNKAIEEIREATQET

SEQ ID NO: 27 - protein, 17 aa, Artificial Sequence
  t-helper cell epitope
SSKTQTHTQQDRPPQPS

SEQ ID NO: 28 - protein, 17 aa, Artificial Sequence
  t-helper cell epitope
QPSTELEETRTSRARHS

SEQ ID NO: 29 - protein, 17 aa, Artificial Sequence
  t-helper cell epitope
RHSTTSAQRSTHYDPRT

SEQ ID NO: 30 - protein, 17 aa, Artificial Sequence
  t-helper cell epitope
PRTSDRPVSYTMNRTRS

SEQ ID NO: 31 - protein, 17 aa, Artificial Sequence
  t-helper cell epitope
TRSRKQTSHRLKNIPVH

SEQ ID NO: 32 - protein, 17 aa, Artificial Sequence
  t-helper cell epitope
TELLSIFGPSLRDPISA

SEQ ID NO: 33 - protein, 17 aa, Artificial Sequence
  t-helper cell epitope
PRYIATNGYLISNFDES

SEQ ID NO: 34 - protein, 17 aa, Artificial Sequence
  t-helper cell epitope
CIRGDTSSCARTLVSGT

SEQ ID NO: 35 - protein, 17 aa, Artificial Sequence
  t-helper cell epitope
DESSCVFVSESAICSQN

SEQ ID NO: 36 - protein, 17 aa, Artificial Sequence
  t-helper cell epitope
TSTIINQSPDKLLTFIA

SEQ ID NO: 37 - protein, 17 aa, Artificial Sequence
  t-helper cell epitope
SPDKLLTFIASDTCPLV

SEQ ID NO: 38 - protein, 24 aa, Artificial Sequence
  t-helper cell epitope
STAPPAHGVTSAPDTRAPGSTAPP

SEQ ID NO: 39 - protein, 19 aa, Artificial Sequence
  t-helper cell epitope
GVTSAPDTRPAPGSTASSL

SEQ ID NO: 40 - protein, 18 aa, Artificial Sequence
  t-helper cell epitope
GVTSAPDTRPAPGSTASL

SEQ ID NO: 41 - protein, 27 aa, Artificial Sequence
  t-helper cell epitope
TAPPAHGVTSAPDTRPAPGSTAPPKKG

SEQ ID NO: 42 - protein, 26 aa, Artificial Sequence
  t-helper cell epitope
STAPPAHGVTSAPDTRPAPGSTAPPK

SEQ ID NO: 43 - protein, 4 aa, Artificial Sequence
  t-helper cell epitope
GVAE

SEQ ID NO: 44 - protein, 10 aa, Artificial Sequence
  t-helper cell epitope
TASGVAETTN

SEQ ID NO: 45 - protein, 16 aa, Artificial Sequence
  t-helper cell epitope
TAKSKKFPSYTATYQF

SEQ ID NO: 46 - DNA, 585 bp, Artificial Sequence
  mCD40 antigen-P30
ctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgattcaacaacttcaccgtcagcttctggctgcgcgtgcccaaggtcagcgccagccacctggag

SEQ ID NO: 47 - protein, 195 aa, Artificial Sequence
  mCD40 antigen-P30
LGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRFNNFTVSFWLRVPKVSASHLE

SEQ ID NO: 48 - DNA, 727 bp, Homo sapiens
atgcacgaggaagaaatatacacctctcttcagtgggatagcccagcaccagacacttaccagaaatgtctgtcttccaacaaatgttcaggagcatgctgtcttgtgatggtgatttcatgtgttttctgcatgggattattaacagcatccattttcttgggcgtcaagttgttgcaggtgtccaccattgcgatgcagcagcaagaaaaactcatccaacaagagagggcactgctaaactttacagaatggaagagaagctgtgcccttcagatgaaatattgccaagccttcatgcaaaactcattaagttcagcccataacagcagtccttgtccaaacaattggattcagaacagagaaagttgttactatgtctctgaaatttggagcatttggcacaccagtcaagagaattgtttaaaggaaggttccacgctgctacaaatagagagcaaagaagaaatggattttatcactggcagcttgaggaagattaaaggaagctatgattactgggtggggttgtctcaggatggacacagcggacgctggctttggcaagatggctcctctccttctcctggcctgttgccagcagagagatcccagtcagctaaccaagtctgtggatacgtgaaaagcaattcccttctttcgtctaactgcagcacgtggaagtattttatctgtgagaagtatgcgttgagatcctctgtctgaa

SEQ ID NO: 49 - protein, 241 aa, Homo sapiens
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 50 - DNA, 1236 bp, Homo sapiens
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaa

SEQ ID NO: 51 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 52 - DNA, 33 bp, artificial
  Oligonucleotide designated CD40-Nterm-FW
cgtggcggccgcctagggcagtgtgttacgtgc

SEQ ID NO: 53 - DNA, 60 bp, artificial
  Oligonucleotide designated CD40-P30-Rev
gggcacgcgcagccagaagctgacggtgaagttgttgaatcgcatccgggactttaaacc

SEQ ID NO: 54 - DNA, 45 bp, artificial
  Oligonucleotide designated P30-Cterm template
agcttctggctgcgcgtgcccaaggtcagcgccagccacctggag

SEQ ID NO: 55 - DNA, 31 bp, artificial
  Oligonucleotide designated P30-Cterm-Rev
cagtccgcggctccaggtggctggcgctgac

SEQ ID NO: 56 - DNA, 18 bp, artificial
  Sequencing oligonucleotide
gcgaatgaattgggacct

SEQ ID NO: 57 - DNA, 23 bp, artificial
  Sequencing oligonucleotide
cttctgagatgagtttttgttcg

(19) United States
(12) Patent Application Publication
(10) Pub. No.: US 2015/0132302 A1
(43) Pub. Date: May 14, 2015

(54) Vaccine and uses thereof

(71) Applicants: The University of Sydney, Sydney, New South Wales (AU); The Sydney Children's Hospitals Network (Randwick and Westmead), Westmead, New South Wales (AU); The Royal Prince Alfred Hospital, Camperdown, New South Wales (AU)

(72) Inventors: Stephen Alexander, Westmead (AU); Yuan Min Wang, Westmead (AU); Guoping Zheng, Northmead (AU); Huiling Wu, Sydney (AU); David Charles Hamlyn Harris, Westmead (AU); Yiping Wang, Westmead (AU)

(21) Appl. No.: 14/398,423

(22) PCT Filed: May 1, 2013
(86) PCT No.: PCT/AU2013/000450

§ 371 Date: Oct. 31, 2014

(30) Foreign Application Priority Data
May 1, 2012 (AU) 2012901774

(51) Int. Cl. C07K 14/705; A61K 35/39; A61K 39/08; A61K 39/00; C07K 16/28; C07K 14/33

(52) U.S. Cl. CPC C07K 14/70578; C07K 16/28; C07K 14/33; A61K 39/08; A61K 39/0005; A61K 35/39; C07K 2319/33; C07K 2317/622; A61K 39/00; A61K 2039/505; C07K 2319/55; A61K 2039/6037; A61K 2039/58; A61K 2039/53
USPC 424/134.1; 536/23.4; 530/387.3; 424/239.1; 424/184.1; 435/320.1

(57) ABSTRACT

The present disclosure provides immunogenic compositions, such as vaccines, including DNA vaccines, and uses thereof, e.g., to suppress or prevent an immune response and/or to treat or prevent an autoimmune disease.

### RELATED APPLICATION DATA

The present application claims priority from Australian Provisional Patent Application No. 2012901774 entitled “Vaccine and Uses Thereof” filed on 1 May 2012. The entire contents of that application are hereby incorporated by reference in its entirety.

### SEQUENCE LISTING

The present application is filed with a Sequence Listing in electronic form. The entire contents of the Sequence Listing are hereby incorporated by reference in its entirety.

### FIELD

The present disclosure relates to immunogenic compositions, such as vaccines, including DNA vaccines, and uses thereof, e.g., to suppress or prevent an immune response and/or to treat or prevent an autoimmune disease.

### BACKGROUND

The immune system plays an essential role in protecting a subject against infections. However, the immune system can also be responsible for diseases in a subject or unwanted immune responses.

For example, autoimmune diseases are caused by immune responses against self-antigens. Autoimmune diseases are a large class of diseases that affect a large proportion of the population, e.g., up to 8.5 million people were afflicted by an autoimmune disease in USA in 1996. This class of disease includes type 1 diabetes, rheumatoid arthritis, multiple sclerosis, Crohn's disease, psoriasis and lupus.

Another disease or condition associated with unwanted immune response is allergy, including asthma. Allergies are generally caused by hypersensitivity of the immune system to an antigen (referred to as an “allergen”). Approximately 50 million people in USA suffer from some form of allergy, with the estimated cost estimated to be nearly US$7 billion.

Unwanted immune responses also result in complications of transplants, e.g., cell, tissue or organ transplants. These complications can result in the transplant being rejected by a subject. To overcome these complications, patients can be maintained for extended periods on immunosuppressant drugs, which can cause their own complications, including an increase in infections.

It will be clear to the skilled person from the foregoing that there is a need in the art for reagents and methods for controlling or suppressing unwanted immune responses in a subject.

### SUMMARY

The present inventors have produced vaccines, e.g., DNA vaccines that induce immune responses against co-stimulatory molecules in the subject. For example, the inventors have produced a vaccine that comprises a region that binds an endocytic receptor on a dendritic cell thereby facilitating its uptake. The vaccine also comprises a co-stimulatory molecule e.g., CD40). Without being bound by any theory or mode of action, the inventors consider that at least some of the co-stimulatory molecule is displayed by the dendritic cell to thereby induce an immune response, e.g., an antibody response. Exemplary DNA vaccines produced by the inventors additionally comprise a T helper epitope to ensure a prolonged immune response. By administering a DNA vaccine encoding a polypeptide as described above, the inventors have shown that they can elicit a humoral immune response comprising production of antibodies that neutralize activity of the co-stimulatory molecule without depleting cells expressing the molecule. The inventors have shown that this humoral immune response suppresses immune responses in a subject, e.g., as evidence by the ability to prevent development of an autoimmune disease and to prevent immune cell proliferation in a mixed lymphocyte reaction. The in vitro and in vivo models used by the inventors demonstrate the applicability of their vaccines and DNA vaccines to the treatment or prevention of any condition characterized by unwanted immune response. These findings provide the basis for vaccines and DNA vaccines for suppressing immune responses in a subject.

The present disclosure provides a recombinant nucleic acid encoding a polypeptide comprising the following linked regions:

(i) a region that binds to a cell surface protein expressed on a dendritic cell; and
(ii) a co-stimulatory molecule or an immunogenic fragment thereof.

In one example, the regions are listed in amino to carboxy order.

In one example, upon administration of the nucleic acid or the polypeptide encoded thereby a neutralizing immune response is induced against the co-stimulatory molecule. In one example, the immune response is an antibody response. In one example, the immune response is non-depleting.

In one example, the region that binds to a cell surface protein mediates internalization (e.g., endocytosis) of the polypeptide into a cell upon binding to the cell surface protein. For example, the region (and other regions linked thereto) is internalized upon binding to the cell surface receptor.

In one example, the region that binds to a cell surface protein is a ligand of the cell surface protein or an antibody or a polypeptide comprising an antibody variable region or an antibody analog.

In one example, the region that binds to a cell surface protein expressed on a dendritic cell comprises a variable region of an antibody that binds to the cell surface protein.

In one example, the region is or comprises a domain antibody (dAb).

In one example, the region that binds to a cell surface protein expressed on a dendritic cell comprises a Fv of an antibody that binds to the cell surface protein. The skilled artisan will understand that a Fv comprises an antibody heavy chain variable region (V_{H}) and an antibody light chain variable region (V_{L}).

In one example, the V_{H} and the V_{L} are in a single polypeptide chain. For example, the region is or comprises a single chain Fv fragment (scFv) or a single chain Fab.

In another example, the V_{L} and V_{H} are in separate polypeptide chains. For example, the region is or comprises:

(i) a diabody;
(ii) a triabody;
(iii) a tetrabody;

### (iv) a Fab;

### (v) a F(ab′)₂; or

### (vi) a Fv.

In one example, the region, variable region or the Fv are chimeric, deimmunized, human, CDR grafted, or humanized.

In one example, the cell surface protein expression on a dendritic cell is selected from the group consisting of a mannose receptor, chemokine receptor CCR1, CD80, CD86, CD11c, DEC-205, a Toll-like receptor (TLR), a c-type lectin domain family member, and a Fcγ receptor (FcγR).

In one example, the cell surface protein expression on a dendritic cell is an endocytic receptor. For example, the cell surface protein is DEC-205, CD207 or DCIR2.

In one exemplary form of the disclosure the cell surface protein expressed on a dendritic cell is DEC-205.

In one exemplary form of the disclosure the cell surface protein expressed on a dendritic cell is DC-SIGN.

In one example, the cell surface protein expression on a dendritic cell is a C-type lectin domain family member. For example, the cell surface protein is CLEC9A or CLEC7A.

In one exemplary form of the disclosure the cell surface protein expressed on a dendritic cell is CLEC9A.

In one example, the co-stimulatory molecule encoded by the nucleic acid is a co-stimulatory molecule expressed by an antigen presenting cell.

For example, the co-stimulatory molecule is a co-stimulatory molecule expressed by a B cell. An exemplary co-stimulatory molecule is selected from the group consisting of CD40, CD80, CD84, CD86, CD150, CD229, B7-H4, programmed death 1 (PD-1), transmembrane activator and CAML-interactor (TACI), B cell-activating factor receptor (BAFF R), B cell maturation protein (BCMA), CD30 ligand, OX40 ligand and NTB-A.

In one exemplary form of the disclosure the co-stimulatory molecule is CD40, e.g., human CD40.

An exemplary immunogenic fragment of the co-stimulatory molecule comprises a region of the molecule that induces production of antibodies in a subject that prevent binding of a ligand that induces co-stimulation to the co-stimulatory molecule. Thus, upon administration of a protein or nucleic acid of the disclosure to a subject, the subject produced antibodies that bind to the immunogenic fragment of the co-stimulatory molecule thereby (which antibodies prevent or reduce binding of a ligand to the co-stimulatory molecule), thereby suppressing immune responses in a subject.

In one example, the recombinant nucleic acid of the disclosure additionally comprises a region encoding a T helper epitope.

Sequences of exemplary T helper epitopes are set forth in any one of SEQ ID NOs: 5-45. In one exemplary form of the disclosure T helper epitope is the P30 epitope of tetanus toxoid (SEQ ID NO: 5).

The present disclosure additionally provides a recombinant nucleic acid encoding a polypeptide comprising the following linked regions (listed in amino to carboxy order):

(i) an antibody variable region that binds specifically to DEC-205; and

### (ii) CD40.

The present disclosure additionally provides a recombinant nucleic acid encoding a polypeptide comprising the following linked regions (listed in amino to carboxy order):

(i) scFv that binds specifically to DEC-205 (e.g., human DEC-205); and

### (ii) CD40.

The present disclosure additionally provides a recombinant nucleic acid encoding a polypeptide comprising the following linked regions (listed in amino to carboxy order):

(i) an antibody variable region that binds specifically to DEC-205 (e.g., human DEC-205);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a scFv that binds specifically to DEC-205 (e.g., human DEC-205);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a human scFv that binds specifically to DEC-205 (e.g., human DEC-205);
(ii) human CD40; and
(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) an antibody variable region that binds specifically to DC-SIGN; and

### (ii) CD40.

The present disclosure additionally provides a recombinant nucleic acid encoding a polypeptide comprising the following linked regions (listed in amino to carboxy order):

(i) scFv that binds specifically to DC-SIGN (e.g., human DC-SIGN); and

### (ii) CD40.

The present disclosure additionally provides a recombinant nucleic acid encoding a polypeptide comprising the following linked regions (listed in amino to carboxy order):

(i) an antibody variable region that binds specifically to DC-SIGN (e.g., human DC-SIGN);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a scFv that binds specifically to DC-SIGN (e.g., human DC-SIGN);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a human scFv that binds specifically to DC-SIGN (e.g., human DC-SIGN);
(ii) human CD40; and
(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) an antibody variable region that binds specifically to CLEC9A; and

### (ii) CD40.

The present disclosure additionally provides a recombinant nucleic acid encoding a polypeptide comprising the following linked regions (listed in amino to carboxy order):

(i) scFv that binds specifically to CLEC9A (e.g., human CLEC9A); and

### (ii) CD40.

The present disclosure additionally provides a recombinant nucleic acid encoding a polypeptide comprising the following linked regions (listed in amino to carboxy order):

(i) an antibody variable region that binds specifically to CLEC9A (e.g., human CLEC9A);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a scFv that binds specifically to CLEC9A (e.g., human CLEC9A);

### (ii) CD40; and

(iii) P30 epitope of tetanus toxoid (SEQ ID NO: 5).

The present disclosure additionally provides a recombinant nucleic acid encoding a polypeptide comprising the following linked regions (listed in amino to carboxy order):

(i) a human scFv that binds specifically to CLEC9A (e.g., human CLEC9A);
(ii) human CD40; and
(iii) P30 epitope of tetanus toxoid (SEQ ID NO: 5).

In one example, the nucleic acid of the disclosure is operably linked to a promoter operable in a mammalian cell.

In one example, the nucleic acid is or comprises DNA.

In one example, the nucleic acid is isolated.

The present disclosure also provides a recombinant polypeptide encoded by the nucleic acid of the disclosure.

In one example, the polypeptide is isolated.

The present disclosure additionally provides an immunogenic composition comprising the recombinant nucleic acid of the disclosure or the recombinant polypeptide of the disclosure and a pharmaceutically acceptable carrier.

The present disclosure additionally provides a DNA vaccine comprising the nucleic acid of the disclosure, wherein the nucleic acid is DNA.

The present disclosure also provides a vaccine comprising the polypeptide of the disclosure or the nucleic acid of the disclosure.

The present disclosure also provides a method of treating or preventing an autoimmune disease in a subject, the method comprising immunizing the subject with the recombinant nucleic acid of the disclosure or the recombinant polypeptide of the disclosure or the immunogenic composition or DNA vaccine or vaccine of the disclosure.

The present disclosure also provides a method of treating or preventing an autoimmune disease, an inflammatory disease or allergy in a subject, the method comprising immunizing the subject with a DNA vaccine comprising the recombinant nucleic acid of the disclosure or the immunogenic composition of the disclosure.

The present disclosure also provides for use of the recombinant nucleic acid of the disclosure or the recombinant polypeptide of the disclosure or the immunogenic composition or DNA vaccine or vaccine of the disclosure in the manufacture of a medicament for treating or preventing an autoimmune disease, an inflammatory disease or allergy.

The present disclosure also provides the recombinant nucleic acid of the disclosure or the recombinant polypeptide of the disclosure or the immunogenic composition or DNA vaccine or vaccine of the disclosure for use in treating or preventing an autoimmune disease, an inflammatory disease or allergy.

In one example, the autoimmune disease is autoimmune nephritis.

The present disclosure also provides method for inducing an immune response against an immune cell of a subject, the method comprising immunizing the subject with the recombinant nucleic acid of the disclosure or the recombinant polypeptide of the disclosure or the immunogenic composition or DNA vaccine or vaccine of the disclosure, wherein the immune response neutralizes activity of the co-stimulatory molecule without depleting cells expressing the co-stimulatory molecule.

The present disclosure also provides for use of the recombinant nucleic acid of the disclosure or the recombinant polypeptide of the disclosure or the immunogenic composition or DNA vaccine or vaccine of the disclosure in the manufacture of a medicament for inducing an immune response against an immune cell of a subject, wherein the immune response neutralizes activity of the co-stimulatory molecule without depleting cells expressing the co-stimulatory molecule.

The present disclosure also provides the recombinant nucleic acid of the disclosure or the recombinant polypeptide of the disclosure or the immunogenic composition or DNA vaccine or vaccine of the disclosure for use in inducing an immune response against an immune cell of a subject, wherein the immune response neutralizes activity of the co-stimulatory molecule without depleting cells expressing the co-stimulatory molecule.

The present disclosure also provides a method for preventing or suppressing an immune response in a subject, the method comprising immunizing the subject with the recombinant nucleic acid of the disclosure or the recombinant polypeptide of the disclosure or the immunogenic composition of the disclosure or the DNA vaccine of the disclosure or the vaccine of the disclosure, wherein the immune response neutralizes activity of the co-stimulatory molecule without depleting cells expressing the co-stimulatory molecule.

The present disclosure also provides for use of the recombinant nucleic acid of the disclosure or the recombinant polypeptide of the disclosure or the immunogenic composition or DNA vaccine or vaccine of the disclosure in the manufacture of a medicament for preventing or suppressing an immune response in a subject, wherein the immune response neutralizes activity of the co-stimulatory molecule without depleting cells expressing the co-stimulatory molecule.

The present disclosure also provides the recombinant nucleic acid of the disclosure or the recombinant polypeptide of the disclosure or the immunogenic composition or DNA vaccine or vaccine of the disclosure for use in preventing or suppressing an immune response in a subject, wherein the immune response neutralizes activity of the co-stimulatory molecule without depleting cells expressing the co-stimulatory molecule.

The present disclosure also provides a method of transplanting a cell, tissue or organ into a subject, the method comprising:

(i) performing the method of the disclosure to thereby suppress and immune response in the subject; and
(ii) subsequently or simultaneously transplanting the cell, tissue or organ into the subject or wherein the method of the disclosure is performed in a subject into who the cell, tissue or organ has been previously transplanted.

The present disclosure also provides a method of transplanting a cell, tissue or organ into a subject, the method comprising:

(i) performing the method of the disclosure to thereby suppress and immune response in the subject; and
(ii) subsequently, transplanting the cell, tissue or organ into the subject.

In one example, the transplant is a bone marrow transplant or a hematopoietic stem cell transplant.

In one example, the transplant is a pancreatic transplant or a pancreatic islet transplant or a lung transplant or a liver transplant. For example, the transplant is a pancreatic islet transplant.

The present disclosure also provides a kit or article of manufacture comprising the recombinant nucleic acid of the disclosure or the recombinant polypeptide of the disclosure or the immunogenic composition or DNA vaccine or vaccine of the disclosure packaged with instructions for use in a method of the disclosure.

### BRIEF DESCRIPTION OF THE DRAWINGS

FIG. 1: Construction and modification of scDEC-CD40 and scControl-CD40 DNA vaccines. Open reading frame for mouse CD40 extracellular domain was fused in frame to the C-terminus of scDEC or scControl and tetanus toxoid T helper epitope P30 was fused in frame to the C-terminus of CD40.

FIG. 2: Anti-CD40 autoantibody level in serum measured by ELISA. (A) Sera were collected from rats either vaccinated (scControl-CD40, n=5 or scDEC-CD40, n=5) or nonvaccinated (n=4) 1 week after the 2nd vaccination and 1 week before Fx1A/CFA immunization. All the sera were diluted by 10× and subjected to ELISA. Absorbance was read at 450 nm corrected against background reading at 570 nm. scDEC-CD40 vaccine induced significantly higher serum anti-CD40 autoantibody as compared to scControl-CD40 and non-vaccinated group (** P<0.01) (B) Serum anti-CD40 autoantibody was measured at weeks 4, 6, 8, 10 and 12 post Fx1A/CFA immunization for 4 groups: con-CD40-HN (rats vaccinated with scControl-CD40 DNA followed by Fx1A immunization, n=5), DEC-CD40-HN (rats vaccinated with scDEC-CD40 DNA followed by Fx1A immunization, n=5), HN (non-vaccinated rats immunized with Fx1A, n=4) and CFA (non-vaccinated rats immunized with CFA, n=3). Serum anti-CD40 autoantibody was significantly higher in the DEC-CD40-HN group than the other 3 groups at all 5 time points (* P<0.05). All results are expressed as mean±SD.

FIG. 3: CD40 DNA vaccination protected against real dysfunction. Urine and serum were collected at weeks 6, 8, 10 and 12 post Fx1A/CFA immunization and urine/serum creatinine and serum albumin were measured. (A) Urine protein:creatinine ratio; wk 8: ** P<0.01 between HN and all the other 3 groups; wk 10 and 12: # P<0.001 between HN and DEC-CD40-HN or CFA groups. (B) Serum albumin (C) Serum creatinine. All results are expressed as mean±SD (* P<0.05; ** P<0.01; # P<0.001)

FIG. 4: CD40 DNA vaccination reduced renal structural injury. Kidney tissues harvested at week 12 post Fx1A/CFA immunization were analyzed with periodic acid-Schiff (PAS) staining. (A) Representative pictures from each group are shown (magnification 200×). (B) Semiquantitative scoring of glomerulosclerosis and tubular atrophy by a blinded observer25. Results are expressed as mean±SD (* P<0.05; ** P<0.01).

FIG. 5: Infiltrating immune cells in renal cortex and glomerular IgG deposition were reduced by scDEC-CD40 vaccination Immunohistochemistry staining was performed to assess the number of CD4+, CD8+ or CD68+ (MΦ) cells in renal cortex at week 12 post Fx1A/CFA immunization and IgG deposition was assessed by immunofluorescence staining. (A) Number of cells in one 200× field is shown (average number counted from 10 of 200× random fields per animal). Values are means±SD (* P<0.05; ** P<0.01). (B) Representative pictures for each staining are shown (magnification 200×). (C) IgG glomerular staining (magnification 400×).

FIG. 6: CTLA-4 mRNA expression in DLN was up-regulated by scDEC-CD40 vaccination whereas CD40 mRNA expression remained unchanged. Expression was normalized against Glyceraldehyde 3-phosphate dehydrogenase (GAPDH). Results are expressed as mean±SD (* P<0.05).

FIG. 7: Serum containing anti-CD40 autoantibody suppressed B cell activation and T proliferation. (A) Splenocytes from normal Lewis rat were cultured for 5 days in the presence of 10% serum from DEC-CD40-HN or CFA group or 1 μg/ml of antimouse/rat CD40 agonist antibody (n=3). At the end of the cell culture, cells were harvested and subjected to flow cytometric analysis. Results are expressed as percentage of CD86+ B cells (gated on live cell population and B cells (OX33+)). (B) CFSE-labelled Lewis T cells were stimulated with irradiated Wistar lymphocytes for 5 days in the presence of 5% serum from con-CD40-HN, DEC-CD40-HN and HN groups respectively (n=3). At the end of the cell culture, cells were harvested and subjected to flow cytometric analysis. Results are expressed as percentage of proliferating cells (CD4+ or CD8+ gated cell population). Values are means±SD (* P<0.05).

FIG. 8: CD40 vaccination did not deplete B cells Immunohistochemical staining of B cells using OX33 was performed on spleen sections collected at week 12 post Fx1A/CFA immunization. Representative pictures demonstrate intact B cell numbers and splenic architecture (magnification 400×).

### KEY TO SEQUENCE LISTING

SEQ ID NO: 1—nucleotide sequence encoding Homo sapiens DEC 205

SEQ ID NO: 2—amino acid sequence of Homo sapiens DEC 205

SEQ ID NO: 3—nucleotide sequence encoding Homo sapiens CD40

SEQ ID NO: 4—amino acid sequence of Homo sapiens CD40

SEQ ID NO: 5—T-helper cell epitope

SEQ ID NO: 6—T-helper cell epitope

SEQ ID NO: 7—T-helper cell epitope

SEQ ID NO: 8—T-helper cell epitope

SEQ ID NO: 9—T-helper cell epitope

SEQ ID NO: 10—T-helper cell epitope

SEQ ID NO: 11—T-helper cell epitope

SEQ ID NO: 12—T-helper cell epitope

SEQ ID NO: 13—T-helper cell epitope

SEQ ID NO: 14—T-helper cell epitope

SEQ ID NO: 15—T-helper cell epitope

SEQ ID NO: 16—T-helper cell epitope

SEQ ID NO: 17—T-helper cell epitope

SEQ ID NO: 18—T-helper cell epitope

SEQ ID NO: 19—T-helper cell epitope

SEQ ID NO: 20—T-helper cell epitope

SEQ ID NO: 21—T-helper cell epitope

SEQ ID NO: 22—T-helper cell epitope

SEQ ID NO: 23—T-helper cell epitope

SEQ ID NO: 24—T-helper cell epitope

SEQ ID NO: 25—T-helper cell epitope

SEQ ID NO: 26—T-helper cell epitope

SEQ ID NO: 27—T-helper cell epitope

SEQ ID NO: 28—T-helper cell epitope

SEQ ID NO: 29—T-helper cell epitope

SEQ ID NO: 30—T-helper cell epitope

SEQ ID NO: 31—T-helper cell epitope

SEQ ID NO: 32—T-helper cell epitope

SEQ ID NO: 33—T-helper cell epitope

SEQ ID NO: 34—T-helper cell epitope

SEQ ID NO: 35—T-helper cell epitope

SEQ ID NO: 36—T-helper cell epitope

SEQ ID NO: 37—T-helper cell epitope

SEQ ID NO: 38—T-helper cell epitope

SEQ ID NO: 39—T-helper cell epitope

SEQ ID NO: 40—T-helper cell epitope

SEQ ID NO: 42—T-helper cell epitope

SEQ ID NO: 43—T-helper cell epitope

SEQ ID NO: 44—T-helper cell epitope

SEQ ID NO: 45—T-helper cell epitope

SEQ ID NO: 46—nucleotide sequence encoding mCD40 antigen

SEQ ID NO: 47—amino acid sequence of mCD40 antigen

SEQ ID NO: 48—nucleotide sequence encoding human CLEC9A

SEQ ID NO: 49—amino acid sequence of human CLEC9A

SEQ ID NO: 50—nucleotide sequence encoding human DC-SIGN

SEQ ID NO: 51: amino acid sequence of human DC-SIGN

SEQ ID NO: 52: nucleotide sequence of oligonucleotide designated CD40-Nterm-FW

SEQ ID NO: 53: nucleotide sequence of oligonucleotide designated CD40-P30-Rev

SEQ ID NO: 54: nucleotide sequence of oligonucleotide designated P30-Cterm template

SEQ ID NO: 55: nucleotide sequence of oligonucleotide designated P30-Cterm-Rev

SEQ ID NO: 56: nucleotide sequence of an oligonucleotide for sequencing

SEQ ID NO: 57: nucleotide sequence of an oligonucleotide for sequencing

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; Benny K. C. Lo, Antibody Engineering: Methods and Protocols, (2004) Humana Press, Vol. 248; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture Practical Approach, Third Edition (John R. W. Masters, ed., 2000), ISBN 0199637970, whole of text.

The term “and/or”, e.g., “X and/or Y” shall be understood to mean either “X and Y” or “X or Y” and shall be taken to provide explicit support for both meanings or for either meaning.

### SELECTED DEFINITIONS

For the purposes of nomenclature and not limitation a sequence of a human DEC-205 encoding nucleic acid is set forth in SEQ ID NO: 1 and a sequence of a human DEC-205 protein is set forth in SEQ ID NO: 2. Additional sequences of DEC-205 are disclosed in NCBI RefSeq NP_{—}002340.2.

For the purposes of nomenclature and not limitation a sequence of a human CLEC9A encoding nucleic acid is set forth in SEQ ID NO: 48 and a sequence of a human CLEC9A protein is set forth in SEQ ID NO: 39. Additional sequences of CLEC9A are disclosed in NCBI RefSeq NP_{—}997228.1.

For the purposes of nomenclature and not limitation a sequence of a human DC-SIGN encoding nucleic acid is set forth in SEQ ID NO: 50 and a sequence of a human DC-SIGN protein is set forth in SEQ ID NO: 51. Additional sequences of DC-SIGN are disclosed in NCBI RefSeq NP_{—}001138365.1, NP_{—}001138366.1, NP_{—}001138367.1, NP_{—}001138368.1, NP_{—}001138369.1, NP_{—}001138371.1 and NP_{—}066978.1.

For the purposes of nomenclature and not limitation a sequence of a human CD40 encoding nucleic acid is set forth in SEQ ID NO: 3 and a sequence of a human CD40 protein is set forth in SEQ ID NO: 4. Additional sequences of CD40 are disclosed in RefSeq NP_{—}001241.1 and NP_{—}690593.1.

As used herein, the term “cell surface protein” will be understood to mean a protein that is displayed on the surface of a cell such that it is capable of being bound by another molecule, such as, an antibody variable region or a Fv. In this regard, the protein can be post-translationally modified (e.g., can be a glycoprotein or a phosphoprotein) and the other molecule can bind to the modification or the protein can be part of a complex and the other molecule can bind to the protein as part of the complex or to the complex. Exemplary cell surface proteins are expressed specifically by a recited cell type (e.g., a dendritic cell) or expressed by the cell type and some additional cells in the subject.

As used herein, the term “co-stimulatory molecule” will be taken to mean a molecule (e.g., a protein) that is expressed by an immune cell (e.g., an antigen presenting cell) that is involved in inducing proliferation of immune cells and activation of an immune response. For example, a B cell binds an antigen with a B cell receptor which induces signal transduction within the B cell and as induces the B cell to engulf the antigen, process it, and present it on cell surface MHC II molecules. The presented antigen is recognized by antigen-specific Th2 cells, which bind to the antigen (and B cell) by a T cell receptor. This binding is followed by synthesis and presentation of CD40L (CD154) on the Th2 cell, which binds to CD40 on the B cell, thus co-stimulating the B cell. Co-stimulatory molecules are an art recognized class of proteins. In one example, the co-stimulatory molecule is a molecule (e.g., a protein) expressed on an antigen presenting cell, e.g., a B cell.

As used herein, the term “endocytic receptor” will be understood to mean a receptor on the surface of a cell, e.g., a dendritic cell, which, upon binding of a ligand or other binding molecule, is endocytosed (i.e., by receptor-mediated endocytosis). Exemplary endocytic receptors on dendritic cells include DEC-205, DC-SIGN, TLR5, CD207 and DCIR2.

As used herein, the term “C-type lectin domain family member” or “CLEC”, will be understood to mean understood to mean a calcium-dependent lectin expressed on the surface of a cell, e.g., a dendritic cell. Exemplary CLECs are CLEC7A and CLEC9A.

As used herein, the term “antigen presenting cell” refers to a recognized class of cells that display antigens as complexes with major histocompatibility complex (MHC) molecules on their surface. In one example, the antigen presenting cells are professional antigen presenting cells, i.e., express MHC class II molecules. In one example, the antigen presenting cell is a dendritic cell, a macrophage or a B cell. In one example, the antigen presenting cell is a B cell.

As used herein, the term “dendritic cell” refers to a sub-type of antigen presenting cells that are characterized at the morphological level by numerous membrane processes that extend out from the main cell body (similar to dendrites on neurons) and at the biochemical level by cell surface expression of MHC class II molecules and lack of expression of one or more of CD3, CD14, CD19, CD56 and/or CD66b. Subsets of dendritic cells express on their cell surface CD11a, CD11c, CD50, CD54, CD58, CD102, CD80 and/or CD86. Some DCs also express toll-like receptors 2, 3, 4, 7 and/or 9. The term “dendritic cell” includes myeloid dendritic cells and plasmacytoid dendritic cells.

As used herein, the term “B cell” will be understood to mean an art-recognized class of immune cells also referred to as “lymphocytes”, which display B cell receptors on their cell surface and/or produce antibodies that bind to antigens and that can act as antigen presenting cells. In some examples, the B cell is a late pro B cell, a large pre B cell, a small pre C cell, an immature B cell or a mature B cell. In some examples, the B cell expresses CD19. In some examples, the B cell is C40 expressing B cell.

As used herein, the term “co-stimulatory molecule expressed on an antigen presenting cell (or B cell)” shall be taken to mean that the co-stimulatory molecule is expressed by the relevant cell and displayed on the surface of the cell. In one example, this term refers to a co-stimulatory molecule that, when activated (or bound to a ligand), induces or enhances proliferation of the cell displaying the molecule. This term does not mean that the co-stimulatory molecule is only expressed on the antigen presenting cell (or B cell), for example, CD40 (a co-stimulatory molecule expressed on antigen presenting cells, including B cells) can be expressed by some T cells.

As used herein, the term “immunogenic composition” means a composition comprising a nucleic acid or protein that, when administered to a subject, elicits an immune response.

As used herein, the term “vaccine” means a composition comprising a nucleic acid or protein that, when administered to a subject, elicits a therapeutic or protective immune response. In this regard, a “therapeutic immune response” refers to the ability of an immunogenic composition or vaccine to elicit an immune response, e.g., a humoral immune response, which serves to reduce symptoms of an autoimmune disease in a subject. The reduction need not be absolute, i.e., the symptoms of the autoimmune disease need not be totally eradicated, if there is a statistically significant improvement compared with a control population of subjects, e.g. subjects suffering from an autoimmune disease and not administered the vaccine or immunogenic composition. A “protective immune response” refers to the ability of an immunogenic composition or vaccine to elicit an immune response, e.g., a humoral immune response, which serves to protect a subject from an autoimmune disease or a relapse of an autoimmune disease. The protection provided need not be absolute, i.e., the autoimmune disease need not be totally prevented or eradicated, if there is a statistically significant improvement compared with a control population of subjects, e.g. subjects at risk of developing an autoimmune disease or relapsing and not administered the vaccine or immunogenic composition.

As used herein, the term “DNA vaccine” means a vaccine comprising DNA encoding an antigen.

As used herein, the term “immunizing” means administering a nucleic acid, polypeptide or composition to a subject in such a manner so as to induce an immune response, such as, a protective immune response or a therapeutic immune response. This term does not mean that the nucleic acid or protein or composition is administered solus (i.e., without other components), for example, the nucleic acid or protein may be administered with an adjuvant to increase the immune response by the subject.

As used herein, the term “immune response” will be taken to mean a response by a subject that involves generation of antibodies that bind to a polypeptide of the disclosure (i.e., an antibody response). This does not exclude generation of a cell-mediated response.

For the purposes for the present disclosure, the term “antibody” includes a protein capable of specifically binding to one or a few closely related antigens (e.g., a cell surface marker on a dendritic cell) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50-70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H}1 which is 330-440 amino acids in length). The light chain variable region is aligned with the variable region of the heavy chain. The antibody heavy chain can comprise 2 or more additional C_{H} domains (such as, C_{H}2, C_{H}3 and the like) and can comprise a hinge region between the C_{H}1 and C_{H}2 constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example, the antibody is humanized, synhumanized, chimeric, CDR-grafted or deimmunized.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (V_{H} or V_{L}) typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol. Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding domain, i.e., capable of specifically binding to an antigen. The V_{H} and the V_{L} which form the antigen binding domain can be in a single polypeptide chain or in different polypeptide chains. Furthermore, an Fv of the disclosure (as well as any protein of the disclosure) may have multiple antigen binding domains which may or may not bind the same antigen. This tem shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a an antibody variable region or a Fv with an antigen means that the interaction is dependent upon the presence of a particular structure (e.g., an antigenic determinant or epitope) on the antigen. For example, an variable region or Fv recognizes and binds to a specific protein structure rather than to proteins generally. If an antibody binds to epitope “A”, the presence of a molecule containing epitope “A” (or free, unlabeled “A”), in a reaction containing labeled “A” and the variable region or Fv, will reduce the amount of labeled “A” bound to the antibody.

As used herein, the term “specifically binds” or “binds specifically” shall be taken to mean that a variable region or Fv (or other binding region) reacts or associates more frequently, more rapidly, with greater duration and/or with greater affinity with a particular antigen or cell expressing same than it does with alternative antigens or cells. For example, a variable region or Fv that specifically binds to an antigen binds that antigen with greater affinity, avidity, more readily, and/or with greater duration than it binds to other antigens. For example, a variable region or Fv binds to a cell surface protein with materially greater affinity than it does to related proteins or other cell surface proteins or to antigens commonly recognized by polyreactive natural antibodies (i.e., by naturally occurring antibodies known to bind a variety of antigens naturally found in humans). It is also understood by reading this definition that, for example, a variable region or Fv that specifically binds to a first antigen may or may not specifically bind to a second antigen. As such, “specific binding” does not necessarily require exclusive binding or non-detectable binding of another antigen, this is meant by the term “selective binding”. In one example, “specific binding” of a variable region or Fv (or other binding region) binds to an antigen, means that the a variable region or Fv (or other binding region) binds to the antigen with an equilibrium constant (K_{D}) of 100 nM or less, such as 50 nM or less, for example 20 nM or less, such as, 15 nM or less or 10 nM or less or 5 nM or less.

As used herein, the term “neutralize” shall be taken to mean that an immune response (e.g., antibodies produced by an immune cell) is capable of reducing or preventing activity of a molecule, e.g., a co-stimulatory molecule. Methods for determining neutralization are known in the art and/or described herein.

As used herein, the term “without depleting cells expressing the co-stimulatory molecule” will be taken to mean that an immune response does not induce death of a significant proportion of cells expressing the co-stimulatory molecule, e.g., by inducing apoptosis or antibody-dependent cell-mediated cytotoxicity (ADCC), or antibody-dependent cell-mediated phagocytosis (ADCP) or complement-dependent cytotoxicity (CDC) in a subject. For example, the immune response does not induce death of 90% or 80% or 70% or 60% or 50% of cells expressing the co-stimulatory molecule.

As used herein, the terms “preventing”, “prevent” or “prevention” include administering a nucleic acid, immunogenic composition or vaccine of the disclosure to thereby stop or hinder the development of at least one symptom of a condition. This term also encompasses treatment of a subject in remission to prevent or hinder relapse.

As used herein, the terms “treating”, “treat” or “treatment” include administering a nucleic acid, immunogenic composition or vaccine of the disclosure to thereby reduce or eliminate at least one symptom of a specified disease or condition.

As used herein, the term “subject” shall be taken to mean any animal, such as, a mammal. In one example, the mammal is a human or non-human primate. In one example, the mammal is a human.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

### Dendritic Cell Binding Region

### Proteins Expressed on Dendritic Cells

As will be apparent from the description herein a polypeptide of the disclosure (e.g., as encoded by a nucleic acid or DNA vaccine of the disclosure) comprises a region that binds to a protein on the surface of a dendritic cell.

In one example, the protein is a human protein expressed on the surface of a human dendritic cell.

In one example, the protein is set out in Table 1.

TABLE 1
Exemplary proteins expressed on dendritic cells.

| | Nucleotide sequence | Amino acid sequence |
|---|---|---|
| Marker | Refseq number | Refseq number |
| CCR1 | NM_001295.2 | NP_001286.1 |
| CD80 | NM_005191.3 | NP_005182.1 |
| CD86 | NM_001206924.1 | NP_001193853.1 |
| | NM_001206925.1 | NP_001193854.1 |
| | NM_006889.4 | NP_008820.3 |
| | NM_175862.4 | NP_787058.4 |
| | NM_176892.1 | NP_795711.1 |
| CD11c | NM_000887.3 | NP_000878.2 |
| DEC-205 | NM_002349.3 | NP_002340.2 |
| DC-SIGN (syn. CD209) | NM_001144893.1 | NP_001138365.1 |
| | NM_001144894.1 | NP_001138366.1 |
| | NM_001144895.1 | NP_001138367.1 |
| | NM_001144896.1 | NP_001138368.1 |
| | NM_001144897.1 | NP_001138369.1 |
| | NM_001144899.1 | NP_001138371.1 |
| | NM_021155.3 | NP_066978.1 |
| TLR5 | NM_003268.5 | NP_003259.2 |
| CD207 | NM_015717.3 | NP_056532.3 |
| DCIR2 | NM_194447.2 | NP_919429.2 |
| | NM_016184.3 | NP_057268.1 |
| | NM_194448.2 | NP_919430.1 |
| | NM_194450.2 | NP_919432.1 |
| CLEC9A | NM_207345.2 | NP_997228.1 |

In one example, the dendritic cell is a Langerhans dendritic cell and the protein is selected from the group consisting of CCR7, CD14, CD83, DEC205, CLEC7A, EpCAM, EMR1, integrin α E, integrin α M, integrin α X or CD207.

In one example, the dendritic cell is a myeloid dendritic cell and the protein is selected from the group consisting of CD1c, integrin α M, integrin α X, TLR1 or TLR6.

In one example, the dendritic cell is a plasmacytoid dendritic cell and the marker is IL-3Rα, CD45R, DEC205, Ly-6G, integrin α X, TLR7 or TLR 9.

In one example, the region of the polypeptide of the disclosure is internalized upon binding to the protein on the surface of the dendritic cell.

In one example, the protein on the surface of the dendritic cell is an endocytic receptor. For example, the protein is CD207, DCIR2, or DEC-205.

In one example, the protein on the surface of the dendritic cell is a CLEC. For example, the protein is CLEC7A or CLEC9A. For example, the protein is CLEC9A.

### Binding Regions

### Antibody Variable Regions

In one example, the binding region is or comprises at least one variable region from an antibody. The binding region can be or comprise one variable region (e.g., a V_{H} or a V_{L} in the case of a dAb) or can comprise two or more variable regions (e.g., a V_{H} and a V_{L} in the case of a scFv or a ScFab). The present disclosure also contemplates an entire antibody (e.g., in the case of a DNA vaccine, this could be expressed from a single expression construct or vector)

The variable regions can be from known antibodies that bind to the protein expressed on a dendritic cell. Exemplary know antibodies bind to the protein expressed on a dendritic cell are set out in Table 2.

TABLE 2
Exemplary antibodies

| Protein | Type of antibody | Source/Reference |
|---|---|---|
| DEC-205 | Human | WO2009/061996 |
| | Mouse monoclonal | Santa Cruz Biotechnology |
| | Mouse monoclonal | Miltenyi Biotec |
| | Mouse monoclonal | Park et al., J. Immunol. Methods, |
| | | 377: 15-22, 2012 |
| | Human monoclonal | Cheong et al., Blood, 116: 3828- |
| | | 3838, 2010 |
| DC-SIGN | dAB | W02010/046337 |
| | Monoclonal | W02005/058244 |
| | Monoclonal | Dakappagari et al., J. |
| | | Immunother., 30: 715-726, 2007 |
| CCR1 | Monoclonal | WO2000/044790 |
| | Monoclonal | WO2000/044789 |
| CD86 | Monoclonal | WO1995/015340 |
| CD80 and CD86 | Monoclonal | WO2004/076488 |
| CLEC9A | Monoclonal | Lahoud et al., J. Immunol., 187: |
| | | 842-850, 2011 |

The nucleic acid encoding the antibody variable region is cloned using standard technologies for use in a nucleic acid or polypeptide of the disclosure.

In another example, an antibody is produced and the sequence encoding one or both variable regions is cloned. Methods for generating antibodies are known in the art and include immunization-based methods and library screening-based methods.

Immunization-Based Methods

To generate antibodies, a protein normally expressed on the surface of the dendritic cell or an epitope bearing fragment or portion thereof or a modified form thereof or nucleic acid encoding same (an “immunogen”), optionally formulated with any suitable or desired adjuvant and/or pharmaceutically acceptable carrier, is administered to a subject (for example, a non-human animal subject, such as, a mouse, a rat, a chicken etc.) in the form of an injectable composition. Exemplary non-human animals are mammals, such as murine animals (e.g., rats or mice). Injection may be intranasal, intramuscular, sub-cutaneous, intravenous, intradermal, intraperitoneal, or by other known route. Optionally, the immunogen is administered numerous times. Means for preparing and characterizing antibodies are known in the art (See, e.g., Antibodies: A Laboratory Manual, Cold Spring Harbor Laboratory, 1988).

Monoclonal antibodies are exemplary antibodies contemplated by the present disclosure. Generally, production of monoclonal antibodies involves, immunizing a subject (e.g., a rodent, e.g., mouse or rat) with the immunogen under conditions sufficient to stimulate antibody producing cells. In some examples, a mouse genetically-engineered to express human antibodies and not express murine antibodies proteins, is immunized to produce an antibody (e.g., as described in PCT/US2007/008231 and/or Lonberg et al., Nature 368 (1994): 856-859). Following immunization, antibody producing somatic cells (e.g., B lymphocytes) are fused with immortal cells, e.g., immortal myeloma cells. Various methods for producing such fused cells (hybridomas) are known in the art and described, for example, in Kohler and Milstein, Nature 256, 495-497, 1975. The hybridoma cells can then be cultured under conditions sufficient for antibody production.

The present disclosure contemplates other methods for producing antibodies, e.g., ABL-MYC technology (as described, for example in Largaespada et al, Curr. Top. Microbiol. Immunol, 166, 91-96. 1990).

Library-Based Methods

The present disclosure also encompasses screening of libraries of antibodies or fragments thereof. Examples of libraries contemplated by this disclosure include naive libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. No. 5,885,793; U.S. Pat. No. 6,204,023; U.S. Pat. No. 6,291,158; or U.S. Pat. No. 6,248,516.

The proteins according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding domains of antibodies are expressed on phage, e.g., as described in U.S. Pat. No. 6,300,064; U.S. Pat. No. 5,885,793; U.S. Pat. No. 6,204,023; U.S. Pat. No. 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding domains displayed by the library with a target antigen (e.g., a dendritic cell or a protein displayed on the surface of a dendritic cell or a soluble form thereof, e.g., an extracellular domain fused to a Fc region of an antibody) and, following washing, eluting those domains that remain bound to the antigen.

Any variable regions or polypeptides comprising same (e.g., scFvs or Fabs or scFabs) identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

Antibody Variable Region Containing Proteins

Single-Domain Antibodies

In some examples, a region that binds to a cell surface protein on a dendritic cell is or comprises a single-domain antibody (which is used interchangeably with the tem “domain antibody” or “dAb”). A single-domain antibody is a single polypeptide chain comprising all or a portion of the variable region of an antibody. In certain examples, a single-domain antibody is a human single-domain antibody (Domantis, Inc., Waltham, Mass.; see, e.g., 056248516; WO90/05144; WO2003/002609 and/or WO2004/058820). In one example, a single-domain antibody consists of all or a portion of the V_{L} or V_{H} of an antibody that is capable of specifically binding to a protein on the surface of a dendritic cell

Diabodies, Triabodies, Tetrabodies

Exemplary a region that binds to a cell surface protein on a dendritic cell is or comprises are diabodies, triabodies, tetrabodies and higher order protein complexes such as those described in WO98/044001 and WO94/007921.

As used herein, the term “diabody” shall be taken to mean a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens.

As used herein, the term “triabody” shall be taken to mean a protein comprising three associated polypeptide chains, each polypeptide chain comprising the structure as set out above in respect of a diabody wherein the V_{H} of one polypeptide chain is associated with the V_{L} of another polypeptide chain to thereby form a trimeric protein (a triabody).

As used herein, the term “tetrabody” shall be taken to mean a protein comprising four associated polypeptide chains, each polypeptide chain comprising the structure set out above in respect of a diabody and wherein the V_{H} of one polypeptide chain is associated with the V_{L} of another polypeptide chain to thereby form a tetrameric protein (a tetrabody).

The skilled artisan will be aware of diabodies, triabodies and/or tetrabodies and methods for their production. The V_{H} and V_{L} can be positioned in any order, i.e., V_{L}-V_{H} or V_{H}-V_{L}. Generally, these proteins comprise a polypeptide chain in which a V_{H} and a V_{L} are linked directly or using a linker that is of insufficient length to permit the V_{H} and V_{L} to associate. Proteins comprising V_{H} and V_{L} associate to form diabodies, triabodies and/or tetrabodies depending on the length of the linker (if present) and/or the order of the V_{H} and V_{L} domains. For example, a linker comprises 12 or fewer amino acids. For example, in the case of polypeptide chains having the following structure arranged in N to C order V_{H}-X-V_{L}, wherein X is a linker, a linker having 3-12 residues generally results in formation of diabodies, a linker having 1 or 2 residues or where a linker is absent generally results in formation of triabodies. In the case of polypeptide chains having the following structure arranged in N to C order V_{L}-X-V_{H}, wherein X is a linker, a linker having 3-12 residues generally results in formation of diabodies, a linker having 1 or 2 residues generally results in formation of diabodies, triabodies and tetrabodies and a polypeptide lacking a linker generally forms triabodies or tetrabodies.

Single Chain Fv (scFv)

The skilled artisan will be aware that scFvs comprise V_{H} and V_{L} regions in a single polypeptide chain. For example, the polypeptide chain further comprises a polypeptide linker between the V_{H} and V_{L} which enables the scFv to form the desired structure for antigen binding (i.e., for the V_{H} and V_{L} of the single polypeptide chain to associate with one another to form a Fv). This is distinct from a diabody or higher order multimer in which variable regions from different polypeptide chains associate or bind to one another. For example, the linker comprises in excess of 12 amino acid residues with (Gly₄Ser)₃ being one of the more favored linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Modified forms of scFv are also contemplated by the present disclosure, e.g., scFv comprising a linker modified to permit glycosylation, e.g., as described in U.S. Pat. No. 6,323,322.

The skilled artisan will be readily able to produce a scFv or modified form thereof comprising a suitable modified V_{L} according to the present disclosure based on the disclosure herein. For a review of scFv, see Plückthun in The Pharmacology of Monoclonal Antibodies, vol. 113, Rosenburg and Moore eds., Springer Verlag, New York, pp. 269-315, 1994.

Deimmunized, Chimeric, Humanized, and Human Binding Regions

The regions that bind to a cell surface protein on a dendritic cell of the present disclosure may be or comprise a humanized.

The term “humanized” in relation to a region shall be understood to refer to a polypeptide or protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized polypeptides or proteins also include polypeptides or proteins in which one or more residues of the human protein are modified by one or more amino acid substitutions and/or one or more FR residues of the human protein are replaced by corresponding non-human residues. Humanized polypeptides or proteins may also comprise residues which are found in neither the human antibody or in the non-human antibody. Any additional regions of the polypeptide or protein or region are generally human. Humanization can be performed using a method known in the art, e.g., U.S. Pat. No. 5,225,539, U.S. Pat. No. 6,054,297, U.S. Pat. No. 7,566,771 or U.S. Pat. No. 5,585,089. The term “humanized protein” also encompasses a super-humanized protein, e.g., as described in U.S. Pat. No. 7,732,578.

The regions that bind to a cell surface protein on a dendritic cell of the present disclosure may be or comprise human polypeptides or proteins. The term “human protein” or “human polypeptide” as used herein refers to polypeptides or proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

In one example, the regions that bind to a cell surface protein on a dendritic cell of the present disclosure is or comprise a chimeric protein or polypeptide. The term “chimeric proteins” or “chimeric polypeptides” refers to proteins or polypeptides in which a variable region is from a particular species (e.g., murine, such as mouse or rat) or belonging to a particular antibody class or subclass, while the remainder of the protein (e.g., a C_{H}1 or CL) is from a protein derived from another species (such as, for example, human or non-human primate) or belonging to another antibody class or subclass. In one example, a chimeric protein or polypeptide comprises a V_{H} and/or a V_{L} from a non-human antibody (e.g., a murine antibody) and the remaining regions of the antibody are from a human antibody. The production of such chimeric proteins or polypeptides is known in the art, and may be achieved by standard means (as described, e.g., in U.S. Pat. No. 6,331,415; U.S. Pat. No. 5,807,715; U.S. Pat. No. 4,816,567 and U.S. Pat. No. 4,816,397).

The present disclosure also contemplates a deimmunized region, e.g., as described in WO2000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein. For example, the region is analyzed to identify one or more B or T cell epitopes and one or more amino acid residues within the epitope is mutated to thereby reduce the immunogenicity of the region.

### Antibody Analogs

Heavy Chain Immunoglobulins

Heavy chain immunoglobulins differ structurally from many other forms of immunoglobulin (e.g., antibodies,), in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “V_{HH} domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble. Heavy chain immunoglobulins and variable regions domains thereof domains derived therefrom can also comprise long surface loops (particularly CDR3), which facilitate penetration of and binding to cavities often found in antigens such as enzymes and on the surface of proteins of viruses and agents causative of infectious diseases.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005/118629.

V-Like Proteins

An example of an antibody variable region analog is a variable region of a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other antibody variable region analogs include proteins with V-like domains, which are generally monomeric. Examples of proteins comprising such V-like domains include CTLA-4, CD28 and ICOS. Further disclosure of proteins comprising such V-like domains is included in WO1999/045110.

Adnectins

In one example, an antibody variable region analog is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

Adnectins are generally monomeric proteins.

Anticalins

In a further example, an antibody variable region analog is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

Affibodies

In a further example, an antibody variable region analog is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

Affibodies are generally monomeric proteins.

DARPins

In a further example, an antibody variable region analog is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Ligands

In another example, a region that binds to a protein on the surface of a dendritic cell is or comprises a ligand of the protein. For example, the ligand is selected from the group consisting of:

DC-SIGN-ICAM-3

CD86-CD28

CD80-CD28

### Co-Stimulatory Molecule

As will be apparent to the skilled artisan a polypeptide of the disclosure comprises a co-stimulatory molecule, e.g., against which an immune response is raised.

In one example, a co-stimulatory molecule is expressed by an antigen presenting cell, such as a B cell.

In one example, the co-stimulatory molecule is different to the cell surface protein to which the region of the polypeptide binds.

Exemplary co-stimulatory molecules and their encoding nucleic acids are set out in Table 3

TABLE 3
Co-stimulatory molecules

| | Nucleotide sequence | Amino acid sequence |
|---|---|---|
| Marker | Refseq number | Refseq number |
| CD40 | NM_001250.4 | NP_001241.1 |
| | NM_152854.2 | NP_690593.1 |
| CD80 | NM_005191.3 | NP_005182.1 |
| CD84 | NM_001184879.1 | NP_001171808.1 |
| | NM_001184881.1 | NP_001171810.1 |
| | NM_001184882.1 | NP_001171811.1 |
| | NM_003874.3 | NP_003865.1 |
| CD86 | NM_001206924.1 | NP_001193853.1 |
| | NM_001206925.1 | NP_001193854.1 |
| | NM_006889.4 | NP_008820.3 |
| | NM_175862.4 | NP_787058.4 |
| | NM_176892.1 | NP_795711.1 |
| CD150 | NM_003037.2 | NP_003028.1 |
| CD229 | NM_001033667.1 | NP_001028839.1 |
| | NM_002348.2 | NP_002339.2 |
| B7-H4 | NM_001253849.1 | NP_001240778.1 |
| | NM_001253850.1 | NP_001240779.1 |
| | NM_024626.3 | NP_078902.2 |
| PD-1 | NM_005018.2 | NP_005009.2 |
| TACI | NM_012452.2 | NP_036584.1 |
| BAFF-R | NM_052945.3 | NP_443177.1 |
| BCMA | NM_001192.2 | NP_001183.2 |
| CD30-ligand | NM_001244.3 | NP_001235.1 |
| | NM_001252290.1 | NP_001239219.1 |
| OX40 ligand | NM_003326.3 | NP_003317.1 |
| NTB-A | NM_001184714.1 | NP_001171643.1 |
| | NM_001184715.1 | NP_001171644.1 |
| | NM_001184716.1 | NP_001171645.1 |
| | NM_052931.4 | NP_443163.1 |

In one example, the polypeptide of the disclosure comprises an immunogenic fragment of a co-stimulatory molecule. In one example, the immunogenic fragment is or comprises a B cell epitope, i.e., capable of eliciting an antibody response when administered to a subject.

Methods of predicting B cell epitopes are known in the art.

In one example, a B cell epitope is predicted using the method described by Hopp, Peptide Research, 6: 183-190 1993, wherein a hydrophilic peptide is selected as it is more likely to occur at the surface of the native protein. However, a peptide should not be too highly charged, as this may reduce the efficiency of antibody generation.

In another example, a B cell epitope is predicted using the method described by Palfreyman et al J. Immunol. Meth. 75, 383-393, 1984, wherein the amino- and/or carboxy-terminal amino acids are used to generate a peptide against which specific antibodies are raised.

In yet another example, a B cell epitope is predicted using an algorithm such as for example that described in Kolaskar and Tongaonkar FEBS Lett. 276(1-2) 172-174, 1990. Such methods are based upon determining the hydrophilicity of regions of a protein, usually 6 amino acids, and determining those hydrophilic regions that are associated with turns in proteins, surface flexibility, or secondary structures, and are unlikely to be modified at the post-translational level, such as, for example by glycosylation. Such regions of a protein are therefore likely to be exposed, that is, at the surface of the three-dimensional structure of the protein. Furthermore, as these regions are not modified, they are likely to remain constant and as such offer a likely site of antibody recognition.

Methods for determining regions or peptides capable of eliciting an immune response useful according to the present disclosure will be apparent to the skilled artisan based on the disclosure herein.

### T Helper Epitopes

As will be apparent from the disclosure herein, some example of the present disclosure comprise or encode a T helper epitope. A T-helper epitope is any T-helper epitope known to the skilled artisan for enhancing an immune response in a particular target subject (i.e. a human subject, or a specific non-human animal subject such as, for example, a rat, mouse, guinea pig, dog, horse, pig, or goat). Exemplary T-helper epitopes comprise at least about 10-24 amino acids in length, more generally about 15 to about 20 amino acids in length.

In one example, the epitope is a promiscuous or permissive T-helper epitope are as these are readily synthesized chemically and obviate the need to use longer polypeptides comprising multiple T-helper epitopes.

Examples of promiscuous or permissive T-helper epitopes suitable are selected from the group consisting of:
(i) a rodent or human T-helper epitope of tetanus toxoid peptide (TTP), such as, for example amino acids 830-843 of TTP (Panina-Bordignon et al., Eur. J. Immun. 19, 2237-2242, 1989) or P30 (amino acids 947-967 or TTP) or P2 (amino acids 830-843 of TTP) (Men et al., Vaccine. 14:1442-1450, 1996);
(ii) a rodent or human T-helper epitope of Plasmodium falciparum pfg27;
(iii) a rodent or human T-helper epitope of lactate dehydrogenase;
(iv) a rodent or human T-helper epitope of the envelope protein of HIV or H1Vgp120 (Berzofsky et al., J. Clin. Invest. 88, 876-884, 1991);
(v) a synthetic human T-helper epitope (PADRE) predicted from the amino acid sequence of known anchor proteins (Alexander et al., Immunity 1, 751-761, 1994);
(vi) a rodent or human T-helper epitope of measles virus fusion protein (MV-F; Muller et al., Mol. Immunol. 32, 37-47, 1995; Partidos et al., J. Gen. Virol., 71, 2099-2105, 1990);
(vii) a T-helper epitope comprising at least about 10 amino acid residues of canine distemper virus fusion protein (CDV-F) such as, for example, from amino acid positions 148-283 of CDV-F (Ghosh et al., Immunol. 104, 58-66, 2001; International Patent Publication No. WO 00/46390);
(viii) a human T-helper epitope derived from the peptide sequence of extracellular tandem repeat domain of MUC1 mucin (US Patent Application No. 0020018806);
(ix) a rodent or human T-helper epitope of influenza virus haemagglutinin (IV-H) (Jackson et al. Virol. 198, 613-623, 1994; and
(x) a bovine or camel T-helper epitope of the VP3 protein of foot and mouth disease virus (FMDV-0₁ Kaufbeuren strain), comprising residues 173 to 176 of VP3 or the corresponding amino acids of another strain of FMDV.

As will be known to those skilled in the art, a T-helper epitope may be recognized by one or more mammals of different species. Accordingly, the designation of any T-helper epitope herein is not to be considered restrictive with respect to the immune system of the species in which the epitope is recognized. For example, a rodent T-helper epitope can be recognized by the immune system of a mouse, rat, rabbit, guinea pig, or other rodent, or a human or dog.

In one example, the T-helper epitope will comprise an amino acid sequence selected from the group consisting of:

(i)
(SEQ ID NO: 5)
FNNFTVSFWLRVPKVSASHLE
(ii)
(SEQ ID NO: 6)
GALNNRFQIKGVELKS from IV-H;
(iii)
(SEQ ID NO: 7)
ALNNRFQIKGVELKS from IV-H;
(iv)
(SEQ ID NO: 8)
LSEIKGVIVHRLEGV from MV-F;
(v)
(SEQ ID NO: 9)
TAAQITAGIALHQSNLN from CDV-F;
(vi)
(SEQ ID NO: 10)
IGTDNVHYKIMTRPSHQ from CDV-F;
(vii)
(SEQ ID NO: 11)
YKIMTRPSHQYLVIKLI from CDV-F;
(viii)
(SEQ ID NO: 12)
SHQYLVIKLIPNASLIE from CDV-F;
(ix)
(SEQ ID NO: 13)
KLIPNASLIENCTKAEL from CDV-F;
(x)
(SEQ ID NO: 14)
LIENCTKAELGEYEKLL from CDV-F;
(xi)
(SEQ ID NO: 15)
AELGEYEKLLNSVLEPI from CDV-F;
(xii)
(SEQ ID NO: 16)
KLLNSVLEPINQALTLM from CDV-F;
(xiii)
(SEQ ID NO: 17)
EPINQALTLMTKNVKPL from CDV-F;
(xiv)
(SEQ ID NO: 18)
TLMTKNVKPLQSLGSGR from CDV-F;
(xv)
(SEQ ID NO: 19)
KPLQSLGSGRRQRRFAG from CDV-F;
(xvi)
(SEQ ID NO: 20)
SGRRQRRFAGVVLAGVA from CDV-F;
(xvii)
(SEQ ID NO: 21)
FAGVVLAGVALGVATAA from CDV-F;
(xviii)
(SEQ ID NO: 22)
GVALGVATAAQITAGIA from CDV-F;
(xix)
(SEQ ID NO: 23)
GIALHQSNLNAQAIQSL from CDV-F;
(xx)
(SEQ ID NO: 24)
NLNAQAIQSLRTSLEQS from CDV-F;
(xxi)
(SEQ ID NO: 25)
QSLRTSLEQSNKAIEEI from CDV-F;
(xxii)
(SEQ ID NO: 26)
EQSNKAIEEIREATQET from CDV-F;
(xxiii)
(SEQ ID NO: 27)
SSKTQTHTQQDRPPQPS from CDV-F;
(xxiv)
(SEQ ID NO: 28)
QPSTELEETRTSRARHS from CDV-F;
(xxv)
(SEQ ID NO: 29)
RHSTTSAQRSTHYDPRT from CDV-F;
(xxvi)
(SEQ ID NO: 30)
PRTSDRPVSYTMNRTRS from CDV-F;
(xxvii)
(SEQ ID NO: 31)
TRSRKQTSHRLKNIPVH from CDV-F;
(xxviii)
(SEQ ID NO: 32)
TELLSIFGPSLRDPISA from CDV-F;
(xxix)
(SEQ ID NO: 33)
PRYIATNGYLISNFDES from CDV-F;
(xxx)
(SEQ ID NO: 34)
CIRGDTSSCARTLVSGT from CDV-F;
(xxxi)
(SEQ ID NO: 35)
DESSCVFVSESAICSQN from CDV-F;
(xxxii)
(SEQ ID NO: 36)
TSTIINQSPDKLLTFIA from CDV-F;
(xxxiii)
(SEQ ID NO: 37)
SPDKLLTFIASDTCPLV from CDV-F;
(xxxiv)
(SEQ ID NO: 38)
STAPPAHGVTSAPDTRAPGSTAPP from MUC-1;
(xxxv)
(SEQ ID NO: 39)
GVTSAPDTRPAPGSTASSL from MUC-1;
(xxxvi)
(SEQ ID NO: 40)
GVTSAPDTRPAPGSTASL from MUC-1;
(xxxvii)
(SEQ ID NO: 41)
TAPPAHGVTSAPDTRPAPGSTAPPKKG from MUC-1;
(xxxviii)
(SEQ ID NO: 42)
STAPPAHGVTSAPDTRPAPGSTAPPK from MUC-1;
(xxxix)
(SEQ ID NO: 43)
GVAE from FMDV-VP3 protein;
(xl)
(SEQ ID NO: 44)
TASGVAETTN from FMDV-VP3 protein (residues 170
to 179);
and
(xli)
(SEQ ID NO: 45)
TAKSKKFPSYTATYQF from FMDV.

### Expression Constructs

The nucleic acid of the present disclosure is, in one example, operably linked to a promoter that is operable in a mammalian cell. Such a construct is referred to as an “expression construct”.

An expression construct can be incorporated into an expression vector.

In one example, the expression vector or expression construct is comprises DNA.

DNA expression constructs/vectors are known in the art and the constructs/vectors of the present disclosure may be comprised of any such a known composition. In one example, the vector/which contains at least a promoter for RNA polymerase transcription, and a transcriptional terminator.

In one example, the promoter is the Rous sarcoma virus (RSV) long terminal repeat (LTR) which is a strong transcriptional promoter. In another example, a promoter is the cytomegalovirus promoter with the intron A sequence (CMV-intA). Other exemplary promoters active in mammalian cells include human elongation factor 1-α promoter (EF1), small nuclear RNA promoters (U1a and U1b), α-myosin heavy chain promoter, Simian virus 40 promoter (SV40), Adenovirus major late promoter, β-actin promoter; hybrid regulatory element comprising a CMV enhancer/β-actin promoter or an immunoglobulin promoter or active fragment thereof.

Exemplary transcriptional terminators include the bovine growth hormone terminator.

In addition, to assist in large scale preparation of a nucleic acid of the disclosure an antibiotic resistance marker can be included in the expression construct/vector. Ampicillin resistance genes, neomycin resistance genes or any other pharmaceutically acceptable antibiotic resistance marker may be used. In one example, the antibiotic resistance gene encodes a gene product for neomycin resistance.

Furthermore, to aid in the high level production of a nucleic acid of the disclosure by fermentation in prokaryotic organisms, it is desirable for the vector to contain an origin of replication and be of high copy number. Any of a number of commercially available prokaryotic cloning vectors provide these benefits. An exemplary vector is a pUC-based vector.

DNA expression vectors which exemplify but in no way limit the present invention are disclosed in WO 94/21797.

In one example, the present disclosure provides a recombinant nucleic acid (or DNA) encoding a polypeptide comprising the following linked regions (listed in amino to carboxy order):

(i) an antibody variable region that binds specifically to DEC-205;

### (ii) CD40; and

(iii) P30 epitope of tetanus toxoid (SEQ ID NO: 5), wherein the nucleic acid (or DNA) is operably linked to a promoter.

The present disclosure additionally provides a recombinant nucleic acid encoding a polypeptide comprising the following linked regions (listed in amino to carboxy order):

(i) a scFv that binds specifically to DEC-205;

### (ii) CD40; and

(iii) P30 epitope of tetanus toxoid (SEQ ID NO: 5), wherein the nucleic acid (or DNA) is operably linked to a promoter.

In one example, the present disclosure provides a recombinant nucleic acid (or DNA) encoding a polypeptide comprising the following linked regions (listed in amino to carboxy order):

(i) an antibody variable region that binds specifically to DC-SIGN;

### (ii) CD40; and

(iii) P30 epitope of tetanus toxoid (SEQ ID NO: 5), wherein the nucleic acid (or DNA) is operably linked to a promoter.

The present disclosure additionally provides a recombinant nucleic acid encoding a polypeptide comprising the following linked regions (listed in amino to carboxy order):

(i) a scFv that binds specifically to DC-SIGN;

### (ii) CD40; and

(iii) P30 epitope of tetanus toxoid (SEQ ID NO: 5), wherein the nucleic acid (or DNA) is operably linked to a promoter.

In one example, the present disclosure provides a recombinant nucleic acid (or DNA) encoding a polypeptide comprising the following linked regions (listed in amino to carboxy order):

(i) an antibody variable region that binds specifically to CLEC9A;

### (ii) CD40; and

(iii) P30 epitope of tetanus toxoid (SEQ ID NO: 5), wherein the nucleic acid (or DNA) is operably linked to a promoter.

The present disclosure additionally provides a recombinant nucleic acid encoding a polypeptide comprising the following linked regions (listed in amino to carboxy order):

(i) a scFv that binds specifically to CLEC9A;

### (ii) CD40; and

(iii) P30 epitope of tetanus toxoid (SEQ ID NO: 5), wherein the nucleic acid (or DNA) is operably linked to a promoter.

The nucleic acids can be codon optimized for expression in the subject to be immunized.

Methods for producing an cloning nucleic acids are known in the art and described, for example, in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). For example, the nucleic acid or a region thereof is isolated or produced using a known method, such as, for example, amplification (e.g., using PCR or splice overlap extension) or isolated from nucleic acid from an organism using one or more restriction enzymes or isolated from a library of nucleic acids.

### DNA Vaccines

In one example, the present disclosure provides a DNA vaccine comprising a nucleic acid of the present disclosure that is in a form expressible in a mammalian cell.

The vector vaccines of the present disclosure may be formulated in any pharmaceutically effective formulation for administration. Any such formulation may be, for example, a saline solution such as phosphate buffered saline (PBS).

It will be useful to utilize pharmaceutically acceptable formulations which also provide long-term stability of the DNA vaccines of the present disclosure. During storage as a pharmaceutical entity, DNA plasmids undergo a physiochemical change in which the supercoiled plasmid converts to the open circular and linear form. A variety of storage conditions (low pH, high temperature, low ionic strength) can accelerate this process. Therefore, the removal and or chelation of trace metal ions (with succinic or malic acid, or with chelators containing multiple phosphate ligands) from the DNA plasmid solution, from the formulation buffers or from the vials and closures, stabilizes the DNA plasmid from this degradation pathway during storage. In addition, inclusion of non-reducing free radical scavengers, such as ethanol or glycerol, are useful to prevent damage of the DNA plasmid from free radical production that may still occur, even in apparently demetalated solutions. Furthermore, the buffer type, pH, salt concentration, light exposure, as well as the type of sterilization process used to prepare the vials, may be controlled in the formulation to optimize the stability of the DNA vaccine. Therefore, formulations that will provide the highest stability of the DNA vaccine will be one that includes a demetalated solution containing a buffer (phosphate or bicarbonate) with a pH in the range of 7-8, a salt (NaCl, KCl or LiCl) in the range of 100-200 mM, a metal ion chelator (e.g., EDTA, diethylenetriaminepenta-acetic acid (DTP A), malate, inositol hexaphosphate, tripolyphosphate or polyphosphoric acid), a non-reducing free radical scavenger (e.g. ethanol, glycerol, methionine or dimethyl sulf oxide) and the highest appropriate DNA concentration in, a sterile glass vial, packaged to protect the highly purified, nuclease free DNA from light. An exemplary formulation which will enhance long term stability of the DNA vaccines of the present disclosure would comprise a Tris-HCl buffer at a pH from about 8.0 to about 9.0; ethanol or glycerol at about 3% w/v; EDTA or DTPA in a concentration range up to about 5 mM; and NaCl at a concentration from about 50 mM to about 500 mM. The use of such stabilized DNA vaccines and various alternatives to this formulation are described in WO 97/40839.

The DNA vaccines of the present disclosure may also be formulated with an adjuvant or adjuvants which may increase immunogenicity of the DNA polynucleotide vaccines of the present disclosure. A number of these adjuvants are known in the art and are available for use in a DNA vaccine, including but not limited to particle bombardment using DNA-coated gold beads, co-administration of DNA vaccines with plasmid DNA expressing cytokines, chemokines, or co-stimulatory molecules, formulation of DNA with cationic lipids or with experimental adjuvants such as saponin, monophosphoryl lipid A or other compounds which increase immunogenicity of the DNA vaccine.

Another adjuvant for use in the DNA vector vaccines of the present invention are one or more forms of an aluminum phosphate-based adjuvant wherein the aluminum phosphate-based adjuvant possesses a molar PO₄/Al ratio of approximately 0.9. An additional mineral-based adjuvant may be generated from one or more forms of a calcium phosphate. These mineral-based adjuvants are useful in increasing humoral responses to DNA vaccination. These mineral-based compounds for use as DNA vaccines adjuvants are disclosed in WO98/35562.

Another adjuvant is a non-ionic block copolymer which shows adjuvant activity with DNA vaccines. The basic structure comprises blocks of polyoxyethylene (POE) and polyoxypropylene (POP) such as a POE-POP-POE block copolymer. Newman et al. (Critical Reviews in Therapeutic Drug Carrier Systems 15: 89-142, 1998) review a class of non-ionic block copolymers which show adjuvant activity.

### Methods of Administration of DNA Vaccines

The DNA vaccines of the present disclosure are administered to a subject by any means known in the art, such as enteral and parenteral routes. These routes of delivery include but are not limited to intramusclar injection, intraperitoneal injection, intravenous injection, inhalation or intranasal delivery, oral delivery, sublingual administration, subcutaneous administration, transdermal administration, transcutaneous administration, percutaneous administration or any form of particle bombardment, such as a biolostic device such as a “gene gun” or by any available needle-free injection device. Exemplary methods of delivery of the DNA vaccines disclosed herein are intramuscular injection, subcutaneous administration and needle-free injection.

The amount of expressible DNA to be introduced to a vaccine recipient will depend on the strength of the transcriptional and translational promoters used in the DNA construct, and on the immunogenicity of the expressed gene product. In general, an therapeutically or prophylactically effective dose of about 1 μg to greater than about 20 mg, and preferably in doses from about 1 mg to about 5 mg is administered directly into muscle tissue. As noted above, subcutaneous injection, intradermal introduction, impression through the skin, and other modes of administration such as intraperitoneal, intravenous, inhalation and oral delivery are also contemplated. It is also contemplated that booster vaccinations are to be provided in a fashion which optimizes the overall immune response.

The aforementioned nucleic acids, when directly introduced into a subject in vivo, express the encoded polypeptide within the animal and in turn induce an immune response within the host to the expressed co-stimulatory molecule.

### Protein-Based Vaccines

The present disclosure also contemplates protein-based vaccines. In this regard, an expression construct (e.g., as described above) is introduced into a mammalian cell. Examples of useful mammalian host cell lines include monkey kidney CV1 line transformed by SV40 (COS-7); human embryonic kidney line (HEK-293 cells); baby hamster kidney cells (BHK); Chinese hamster ovary cells (CHO); African green monkey kidney cells (VERO-76); or myeloma cells (e.g., NS/0 cells). Bacterial, yeast or insect cell-based expression is also contemplated by the present disclosure.

Standard methods of peptide purification are employed to obtain an isolated polypeptide, including but not limited to various high-pressure (or performance) liquid chromatography (HPLC) and non-HPLC polypeptide isolation protocols, such as size exclusion chromatography, ion exchange chromatography, hydrophobic interaction chromatography, mixed mode chromatography, phase separation methods, electrophoretic separations, precipitation methods, salting in/out methods, immunochromatography, and/or other methods.

Formulation of a polypeptide to be administered will vary according to the route of administration and formulation (e.g., solution, emulsion, capsule) selected. An appropriate pharmaceutical composition comprising a polypeptide to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringers or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate.

The optimum concentration of the polypeptide in the chosen carrier can be determined empirically, according to procedures well known to the skilled artisan, and will depend on the ultimate pharmaceutical formulation desired.

The dosage ranges for the administration of the polypeptide of the disclosure are those large enough to produce the desired effect.

The dosage should not be so large as to cause adverse side effects, such as hyper viscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication. Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

### Conditions to be Treated or Prevented

In one example, a nucleic acid or polypeptide of the disclosure is used to treat an autoimmune disease.

The term “autoimmune disease” refers to a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

Exemplary autoimmune diseases include rheumatologic disorders (such as, for example, rheumatoid arthritis, Sjogren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease. Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)).

In one example, the autoimmune disease is autoimmune nephritis. For example, the autoimmune disease is human membranous nephropathy.

In one example, the disclosure provides a method for treating a subject suffering from an active autoimmune disease. A subject with “active” autoimmune disease is experiencing at least one symptom of autoimmune disease at the time of screening or treatment (e.g., initial treatment).

In a further example, performing a method described herein according to any example may result in disease remission. In one example, the time to disease remission is less than that achieved in a subject who is not treated by performing a method described herein. In another example, the duration of remission is greater than that achieved in a subject who is not treated by a method described herein. For example, the duration of remission may be for at least 24 weeks, such as for at least 48 weeks.

By “disease remission” is intended substantially no evidence of the symptoms of disease. Remission may be achieved within a specified time frame, such as within or at about 8 weeks, from the start of treatment with, or from the initial dose of the vaccine or DNA vaccine. Remission may also be sustained for a period of time, such as for ≧24 weeks, or ≧48 weeks.

In one example, the disclosure provides a method of preventing an autoimmune disease. For example, the method comprises treating a subject who suffers from an autoimmune disease, however is currently in remission to thereby prevent relapse of the autoimmune disease.

### Immune Response Suppression

The present disclosure also provides a method for suppressing or preventing an immune response in a subject. Such a method is useful, for example, for preventing an immune response against a grafted cell, tissue or organ.

Exemplary cell transplants are stem cell transplants (e.g., hematopoietic and/or mesenchymal stem cell transplantation) or pancreatic β islet cell transplants.

Exemplary tissue transplants include, for example, bone marrow transplants, pancreatic islet transplants, blood vessel transplant, heart valve transplant or skin grafts.

Exemplary organ transplants include, for example, pancreatic transplant, lung transplant, liver transplant, heart transplant, kidney transplant or stomach transplant.

In one example, the nucleic acid or polypeptide of the disclosure is administered before the transplant.

In one example, the nucleic acid or polypeptide of the disclosure is administered at the time of or after the transplant.

### Screening Assays

Nucleic acids and their encoded polypeptides of the disclosure are readily screened for biological activity, e.g., as described below.

### Immunogenicity

In one example, a nucleic acid or polypeptide of the disclosure is tested for immunogenicity. For example, the nucleic acid or polypeptide is administered to a subject (e.g., a non-human mammal). After sufficient time to induce an immune response, an antibody containing sample is taken from the subject (e.g., a blood sample). The sample (or a fraction thereof, e.g., plasma or serum) is then brought into contact with the polypeptide, or a polypeptide encoded by the nucleic acid or the co-stimulatory molecule and the level of antibody bound to the polypeptide/molecule detected using standard technology, e.g., ELISA or FLISA (e.g., as described in Scopes (In: Protein purification: principles and practice, Third Edition, Springer Verlag, 1994)). An increase in the level of antibody in a sample from a subject administered (or immunized with) the nucleic acid or polypeptide compared to a subject not administered the nucleic acid or polypeptide is indicative of immunogenicity.

### Binding to a Dendritic Cell

A region of a polypeptide that binds to a protein on a dendritic cell can be tested for binding activity using an antigen binding assay, e.g., as generally described in Scopes (In: Protein purification: principles and practice, Third Edition, Springer Verlag, 1994). Such a method generally involves labeling the polypeptide and contacting it with immobilized antigen or cell expressing same (e.g., a dendritic cell). Following washing to remove non-specific bound protein, the amount of label and, as a consequence, bound protein is detected. Alternatively, or additionally, surface plasmon resonance assays can be used.

By detecting uptake of the label on the compound by a dendritic cell (e.g., by microscopy), a polypeptide that is internalized by a dendritic cell is also determined.

### Neutralization

Nucleic acids and polypeptides of the disclosure can also be tested for their ability to induce an immune response that neutralizes activity of a co-stimulatory molecule.

Various assays are known in the art for assessing the ability of antibodies to neutralize signaling of a ligand through a co-stimulatory molecule.

For example, sera from a subject immunized with a nucleic acid or polypeptide of the disclosure are contacted to a B cell in the presence of a labeled ligand of the co-stimulatory molecule. Following washing to remove unbound ligand, the amount of label bound to the B cell is assessed. A reduced level of label bound to a B cell contacted in the presence of the sera as compared to in the absence of sera (or in the presence of sera from an un-immunized subject) indicates that the antibodies induced by the nucleic acid or polypeptide are neutralizing.

In another example, a mixed lymphocyte reaction is used to assess neutralization activity. Such a method involves culturing a mixture of cells, e.g., lymphocytes depleted of antigen presenting cells and allotypically different antigen presenting cells (or lymphocytes) are cultured together in the presence or absence of serum from a subject immunized with a nucleic acid or polypeptide of the disclosure. Several measures may then be performed to measure activation of immune cells, e.g., cell proliferation is then measured using a standard method, e.g., ¹³H thymidine incorporation; and/or cytokine secretion by T cells. A suppression in T cell proliferation and/or cytokine secretion in the cells cultured in the presence of serum from an immunized subject indicates (compared to cells cultured in the absence of serum) that the serum contains neutralizing antibodies.

Alternatively, or in addition an in vitro method for determining the effect of sera is a 5,6-carboxy fluorescein diacetate succinimidyl ester (CFSE) suppressor assay, e.g., as described herein in the examples. In such an assay, lymphocytes depleted of antigen presenting cells are labeled with CFSE. CFSE labeled cells are then cultured with irradiated PBMCs or lymphocytes in the presence of varying amounts of serum from a subject immunized according to the present disclosure. After a sufficient time, proliferation of the CFSE labeled T cells is analyzed by flow cytometry. Each CFSE signal peak represents one division cycle. The ability of serum to suppress cell proliferation is assessed by comparing CFSE signal peaks of CFSE labeled T cells with and without the presence of serum.

### Depletion

In one example, an immune response induced by a nucleic acid or polypeptide of the disclosure does not deplete cells, e.g., cells expressing the co-stimulatory molecule.

Depletion can be determined, for example, by determining the number of the cells of interest (e.g., B cells) in a sample from a subject immunized with the nucleic acid or polypeptide compared to in a sample from a subject not so immunized.

Other assays for assessing depletion include assays for detecting ADCC induced by antibodies in the serum of a subject immunized with the nucleic acid or protein. In one example, the level of ADCC activity is assessed using a ⁵¹Cr release assay, a europium release assay or a ³⁵S release assay. In each of these assays, cells expressing the co-stimulatory molecule are cultured with one or more of the recited compounds for a time and under conditions sufficient for the compound to be taken up by the cell. In the case of a ³⁵S release assay, the cells can be cultured with ³⁵S-labeled methionine and/or cysteine for a time sufficient for the labeled amino acids to be incorporated into newly synthesized proteins. Cells are then cultured in the presence or absence of the serum and in the presence of immune effector cells, e.g., peripheral blood mononuclear cells (PBMC) and/or NK cells. The amount of ⁵¹Cr, europium and/or ³⁵S in cell culture medium is then detected, and an increase in the presence of the serum compared to in the absence of serum indicates that the serum is depleting. Exemplary publications disclosing assays for assessing the level of ADCC induced by antibodies include Hellstrom, et al. Proc. Natl. Acad. Sci. USA 83:7059-7063, 1986 and Bruggemann, et al., J. Exp. Med. 166:1351-1361, 1987. The level of ADCC induced by an immunoglobulin or antibody include ACTI™ nonradioactive cytotoxicity assay for flow cytometry (CellTechnology, Inc. CA, USA) or CytoTox 96® non-radioactive cytotoxicity assay (Promega, WI, USA).

To assess complement activation, a CDC assay may be performed (see, for example, Gazzano-Santoro et al, J. Immunol. Methods 202: 163, 1996.

### In Vivo Disease Models

A nucleic acid or polypeptide of the disclosure invention can also be administered to an animal model of a disease to test its efficacy. Exemplary animal models of autoimmune disease include
NOD mice to test the ability to suppress, prevent, treat or delay diabetes (e.g., as described in Tang et al. J. Exp. Med., 199: 1455-1465, 2004);
A mouse model of psoriasis (e.g., Wang et al. J Clin Invest. 118(7): 2629-2639, 2008);
Models of rheumatoid arthritis e.g., a SKG strain of mouse (Sakaguchi et al. Nature, 426: 454-460), rat type II collagen arthritis model, mouse type II collagen arthritis model or antigen induced arthritis models in several species (Bendele J Musculoskel Neuron Interact; 1(4):377-385, 2001);
A model of multiple sclerosis (for example, experimental autoimmune encephalomyelitis (EAE; Bradl and Linington Brain Pathol., 6:303-311, 1996);
A model of inflammatory bowel disease (e.g., dextran sodium sulphate (DSS)-induced colitis or Muc2 deficient mouse model of colitis (Van der Sluis et al. Gastroenterology 131: 117-129, 2006); or
A model of autoimmune nephritis, e.g., as described herein.

### Kits

Another example of the disclosure provides kits a nucleic acid, polypeptide, vaccine or DNA vaccine of the disclosure as described above.

In one example, the kit comprises (a) a container comprising a nucleic acid, polypeptide, vaccine or DNA vaccine of the disclosure, optionally in a pharmaceutically acceptable carrier or diluent; and (b) a package insert with instructions for immunizing a subject (e.g., a subject suffering from an autoimmune disease or about to undergo a transplant).

In accordance with this example of the disclosure, the package insert is on or associated with the container. Suitable containers include, for example, bottles, vials, syringes, etc. The containers may be formed from a variety of materials such as glass or plastic. The label or package insert indicates that the composition is used for treating a subject eligible for treatment, e.g., one suffering from an autoimmune disease or about to undergo a transplant, with specific guidance regarding dosing amounts and intervals treatment being provided. The kit may further comprise an additional container comprising a pharmaceutically acceptable diluent buffer, such as bacteriostatic water for injection (BWFI), phosphate-buffered saline, Ringers solution, and/or dextrose solution. The kit may further include other materials desirable from a commercial and user standpoint, including other buffers, diluents, filters, needles, and syringes.

The kit optionally further comprises a container comprises a second medicament, wherein the nucleic acid, polypeptide, vaccine or DNA vaccine of the disclosure is a first medicament, and which kit further comprises instructions on the package insert for treating the subject with the second medicament, in an effective amount. The second medicament may be a compound used to treat autoimmune disease and/or to reduce inflammation at the site of immunization with the nucleic acid, polypeptide, vaccine or DNA vaccine of the disclosure.

The present disclosure includes the following non-limiting examples.

### EXAMPLES

### Example 1

### Materials and Methods

### 1.1 Experimental Animals

Inbred male Lewis rats (aged 6 weeks and weighing 180-200 g), inbred male Wistar rats and outbred male Sprague-Dawley rats (aged 8 weeks and weighing 200-220 g) were purchased from the Animal Resources Centre in Perth, Australia and maintained under standard sterile conditions in the Department of Animal Care at Westmead Hospital. Experiments were carried out in accordance with protocols approved by the Animal Ethics Committee of Sydney West Area Health Service.

### 1.2 Antibodies

Antibodies used in immunohistochemistry included: mouse anti-rat CD4 (OX35) (Serotec, Oxford, UK), mouse anti-rat CD8a (OX8) (eBioscience, CA), mouse anti-rat CD68 (ED1) (Serotec, Oxford, UK), mouse anti-rat CD45RA (OX33) (BD Pharmingen™, San Diego, Calif.) and biotinylated goat anti-mouse immunoglobulin (Zymed Laboratories, San Francisco, Calif.). FITC-goat anti-rat IgG used for immunofluorescence staining was from Zymed Laboratories, San Francisco, Calif. APC-mouse anti-rat CD4 and PE-mouse anti-rat CD8 used for flow cytometry staining were purchased from BD Pharmingen™ (San Diego, Calif.). APC-mouse anti-rat CD45RA (OX33) used for flow cytometry staining was from eBioscience, CA. Anti-mouse/rat CD40 antibody (clone: HM40-3) (eBioscience, CA) was used for in vitro stimulation experiments.

### 1.3 Construction and Modification of the CD40 DNA Vaccine

pSC-DEC-OLLA and pSC-GL117-OLLA vectors used to prepare vaccines. Mouse cDNA for amplification of CD40 was reverse-transcribed from total RNA extracted from spleen of AN mouse. Total RNA was extracted using RNeasy Mini kit (QIAGEN, Hilden, Germany) and reverse transcription was performed using SuperScript™ III First-Strand Synthesis system (Invitrogen, Carlsbad, Calif.). Gene encoding the extracellular domain of CD40 was modified by fusing in frame to sequence encoding P30 epitope (FNNFTVSFWLRVPKVSASHLE (SEQ ID NO: 5)), with P30 at the C-terminus of CD40 (SEQ ID NO: 47). Construction of CD40-P30 fusion DNA was carried out using sequence overlapping primer extension PCR with specific primers: CD40-Nterm-FW: 5′-CGTGGCGGCCGCCTAGGGCAGTGTGTTACGTGC-3′ (SEQ ID NO: 52); CD40-P30-Rev: 5′-GGGCACGCGCAGCCAGAAGCTGACGGTGAAGTTGTTGAAT CGCATCCGGGACTTTAAACC-3′ (SEQ ID NO: 53); P30-Cterm template: 5′-AGCTTCTGGCTGCGCGTGCCCAAGGTCAGCGCCAGCCACCTGGAG-3′ (SEQ ID NO: 54); P30-Cterm-Rev: 5′-CAGTCCGCGGCTCCAGGTGGCTGGCGCTGAC-3′. The CD40-P30 fusion DNA fragment was then cloned into pSC-DEC-OLLA or pSC-GL117-OLLA vector to generate scDEC-CD40 or scControl-CD40 DNA vaccines. The sequences of the two CD40 vaccines were confirmed by DNA sequencing using specific primers: FW 5′-GCGAATGAATTGGGACCT-3′ (SEQ ID NO: 55) and Rev 5′-CTTCTGAGATGAGTTTTTGTTCG-3′ (SEQ ID NO: 56). Plasmid DNA was prepared in large-scale using Plasmid Maxi Prep (QIAGEN, Hilden, Germany).

### 1.4 DNA Vaccination and Induction of Active HN

Rats were divided into 4 groups: DEC-CD40-HN group (n=5): rats vaccinated with scDEC-CD40 followed by Fx1A immunization; con-CD40-HN group (n=5): rats vaccinated with scControl-CD40 followed by Fx1A immunization; HN group (n=4): non-vaccinated rats with Fx1A immunization; CFA group (n=3): non-vaccinated rats with CFA immunization. Rats were immunized with DNA by intramuscular (i.m.) injection in conjunction with electroporation in the anterior tibialis (TA) muscles of the right hind leg. One week before DNA immunization, rats were injected i.m. with 0.75% bupivacaine (1 μl/g body wt; Sigma, St. Louis, Mo.), which was followed by two i.m. DNA injections (3 weeks apart with 300 μg/150 μl sterile H₂O/rat/injection) at the same location as bupivacaine injection. Each DNA injection was followed immediately by square wave electroporation at the injection site using BTX830 two needle array electrodes (BTX Harvard Apparatus, San Diego, Calif.). The distance between the electrodes was 10 mm and the array was inserted longitudinally relative to the muscle fibers. In vivo electroporation parameters were: 100 V/cm; 50-msec pulse length; 6 pulses with reversal of polarity after 3 pulses. Two weeks after the second DNA injection, HN was induced. To induce HN, Lewis rats were immunized subcutaneously (s.c.) in both hind footpads. Each footpad was injected with 100 μl of emulsion containing 15 mg of Fx1A, 1 mg mycobacterium tuberculosis HRa37 (Difco, Detroit, Mich.), 100 μl of IFA (Sigma-Aldrich, St. Louis, Mo.), and 100 μl of PBS. The CFA control rats were immunized with emulsion without Fx1A. Fx1A was extracted from outbred male Sprague-Dawley rats substantially as described previously (Wu et al., J Immunol 171: 4824-4829, 2003). All procedures were performed with rats under isoflurane anesthesia.

1.5 Renal function

Blood and 16-h urine samples were collected every two weeks after the induction of HN. Urine protein concentration was measured using colorimetric assay (Bio-Rad, Hercules, Calif.) based on the method of Bradford. Urine creatinine, serum albumin and serum creatinine were analyzed using an automated chemistry analyzer VITROS (Ortho Clinical Diagnostics, Johnson & Johnson) by staff at the Institute of Clinical Pathology and Medical Research at Westmead Hospital.

### 1.6 Renal Histology and Immunohistochemistry

Coronal sections of the kidney were fixed in 10% neutral-buffered formalin and embedded in paraffin. 4 μm section of paraffin block was stained with periodic acid-Schiff's (PAS) reagent and counterstained with haematoxylin. Renal histopathology was graded as previously described²⁵. Immunohistochemical staining was performed to determine the infiltrations of CD4+, CD8+ T cells and macrophages in the kidney. For staining of CD4+ T cells, frozen sections (cut at 5 μm from kidney tissues embedded in OCT compound) were used. And for staining of CD8+ T cells and macrophages, paraffin sections were used. Sections were incubated with primary antibody (16 h, 4° C.) followed by secondary antibody incubation (30 min, RT) substantially as previously described (Wang et al., J Am Soc Nephrol 17: 697-706, 2006).

Immune cell infiltration was quantified by counting 10 consecutive high power fields per animal and expressed as cells per 200× field. Slides were scanned using ScanScope digital slide scanner (Aperio Technologies, Inc. Vista, Calif.). Image analysis was performed using Image J software (NIH, Bethesda, Md.) Immunofluorescence staining of IgG in kidney sections was performed to assess IgG deposition on glomeruli. Frozen sections were incubated with goat serum (15 mins, RT) followed by FITC goat anti-rat IgG antibody (2 h, RT). Images (magnification 400×) were taken using DeltaVision core microscope (Applied Precision, Inc, Washington).

### 1.7 Mixed Lymphocyte Reaction (MLR)

Lewis rat lymphocytes from spleen were depleted of APC using anti-rat MHC Class II MicroBeads (Miltenyi Biotec GmbH, Bergisch Gladbach, Germany), followed by CFSE staining according to manufacturer's protocol (Invitrogen, Carlsbad, Calif.). CFSE-labelled Lewis lymphocytes (2×10⁵) (responder cells) were stimulated with irradiated (25 Gy 137Cs) unlabelled Wistar lymphocytes (4×10⁵) in flat-bottomed 96-well plates. Cells were cultured in RPMI1640 medium (Lonza, Basel, Switzerland) supplemented with 10% FBS, 100 U/ml Penicillin/streptomycin, 10 mM Hepes, sodium pyruvate, nonessential amino acids (NEAA), 50 μM 2-ME, 2 mM L-glutamine (Invitrogen, Carlsbad, Calif.) and 20 u/ml IL-2 at 37° C., 5% CO₂. After 5 days of culture, cells were harvested and subjected to flow cytometry analysis.

### 1.8 Flow Cytometry Analysis

Single cell suspension was directly stained with APC- and PE-conjugated mouse anti-rat mAb to cell surface antigens. All samples were analyzed on FACSCanto™ flow cytometer (Becton Dickinson, Mountain View, Calif.). Flowjo software was used for analysis (Tree Star, Inc. Ashland, Oreg.).

### 1.9 Real-Time PCR

Total RNA was isolated from rat DLNs and reverse transcribed (as above). cDNA was subjected to quantitative PCR analysis using Taqman® Gene Expression Assays specific for the genes of interest (Applied Biosystem, Australia). PCR reaction mix was subjected to Rotor-Gene 3000 thermal cycler (Corbett Life Science) for acquisition and analysis.

### 1.10 Statistical Analysis

Statistical analysis was performed using one-way analysis of variance (ANOVA) for multiple comparisons. For comparison between two groups, student t-test was performed. Results are expressed as the group mean±SD. Differences were considered significant at P<0.05. Statistical analysis was performed using Prism 5 (GraphPad Software).

### Example 2

### Results

2.1 Construction and Modification of scDEC-CD40 and scControl-CD40 DNA Vaccines

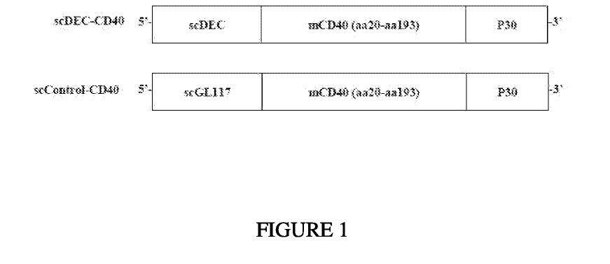

To generate DC-targeted CD40 DNA vaccine, the open reading frame for mouse CD40 extracellular domain was cloned into pSC-DEC-OLLA vector (scDEC), a modified pcDNA3.1 vector that contains a gene encoding single-chain antibody specific for mouse DEC205. A fusion DNA construct was therefore generated with the CD40 gene fused in frame to the C-terminus of scDEC, as shown in FIG. 1. The tetanus toxoid T helper epitope P30 was included at the C-terminus of scDEC-CD40. As a vaccine control, a non-DC-targeted CD40 DNA vaccine (scControl-CD40) was generated as described above with the use of pSC-GL117-OLLA (scControl) vector instead of scDEC vector.

2.2 scDEC-CD40 DNA Vaccination Induces Stronger Anti-CD40 Autoantibody Responses than scControl-CD40 DNA Vaccination

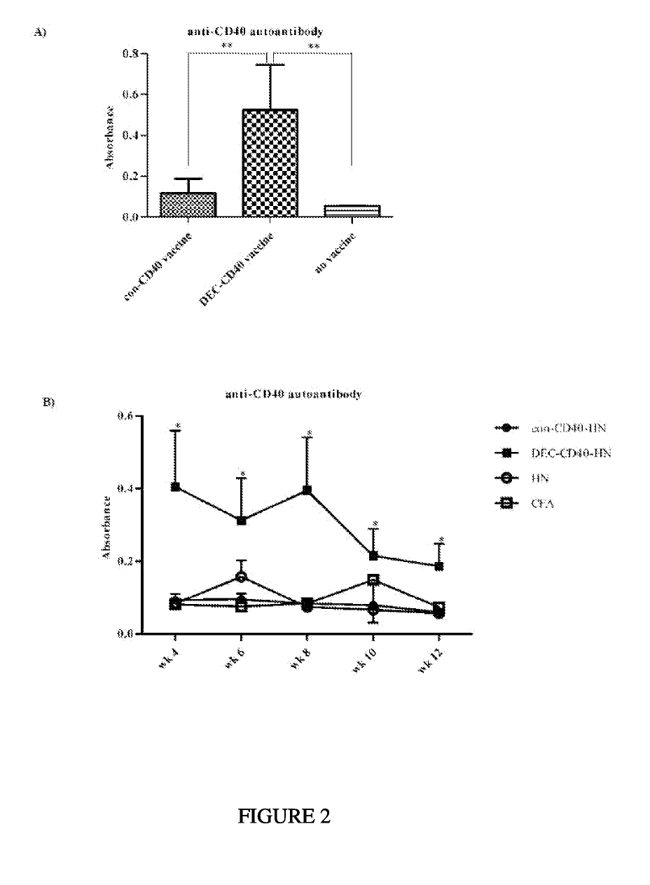

Serum anti-CD40 autoantibody levels were measured to assess the potency of the different vaccines. Rats were injected intramuscularly (i.m.) twice with scDEC-CD40 or scControl-CD40 DNA vaccines with a 3-week interval. One week after the second vaccination, sera were collected and subjected to ELISA analyses. Serum collected from non-vaccinated rats was used as control. As shown in FIG. 2A, there was a 4-fold increase in anti-CD40 autoantibody level in scDEC-CD40 vaccinated rats as compared to scControl-CD40 vaccinated ones (0.52±0.22 vs 0.12±0.07, p<0.01). The anti-CD40 autoantibody level in scControl-CD40 vaccinated rats was not significantly increased compared to that in non-vaccinated rats.

Anti-CD40 autoantibody was measured at 4, 6, 8, 10 and 12 weeks after induction of HN. Rats were divided into 4 groups as described in materials and methods: DEC-CD40-HN, con-CD40-HN, HN and CFA. CD40 antibody levels remained significantly elevated throughout the time course of HN in the DEC-CD40-HN group (FIG. 2B). The anti-CD40 autoantibody level in the con-CD40-HN, HN and CFA groups were low and not significantly different throughout the time course of HN.

### 2.3 CD40 DNA Vaccination Protects Renal Function

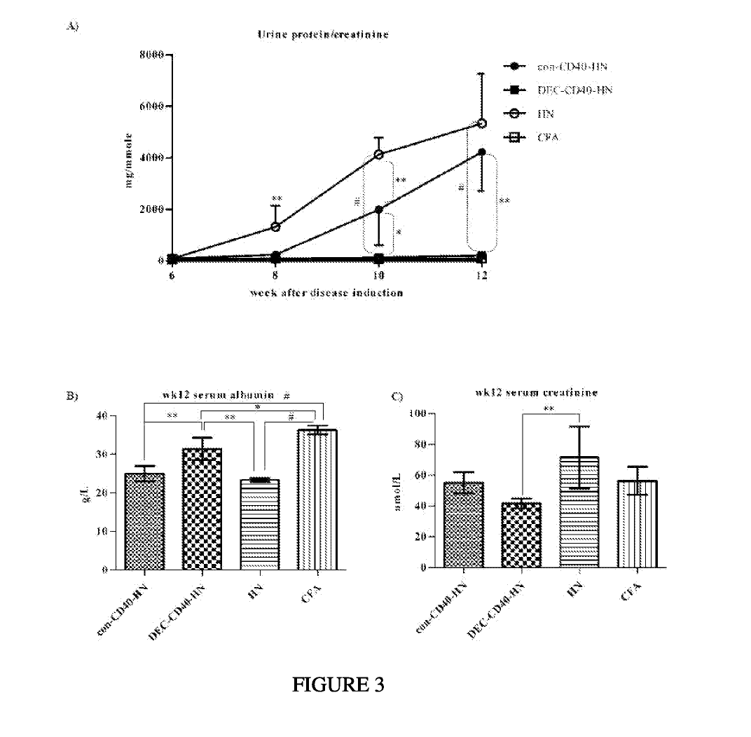

To assess the effects of CD40 vaccination on the development of HN, total urinary protein excretion was measured over 16 hours, urine creatinine, serum albumin and serum creatinine 6, 8, 10, 12 weeks post Fx1A/CFA immunization. The DEC-CD40-HN group did not develop proteinuria throughout the 12 week period. Proteinuria (urine protein/creatinine) of the DEC-CD40-HN group was equivalent to the control CFA group throughout the time-course (FIG. 3A). In the con-CD40-HN group the onset of proteinuria was delayed by 2 weeks as compared to HN group, demonstrating an effect though less potent than with the DEC modified vaccine. At week12, the DEC-CD40-HN group had a significantly higher serum albumin than the con-CD40-HN and HN groups (FIG. 3B) and the serum creatinine level at week 12 was significantly lower in DEC-CD40-HN group than HN group (FIG. 3C). The results show that both scControl-CD40 vaccination and scDEC-CD40 vaccination protects HN rats with reduction in proteinuria however scDEC-CD40 vaccination was significantly more protective than scControl-CD40 vaccination.

### 2.4 CD40 DNA Vaccination Reduces Renal Structural Injury

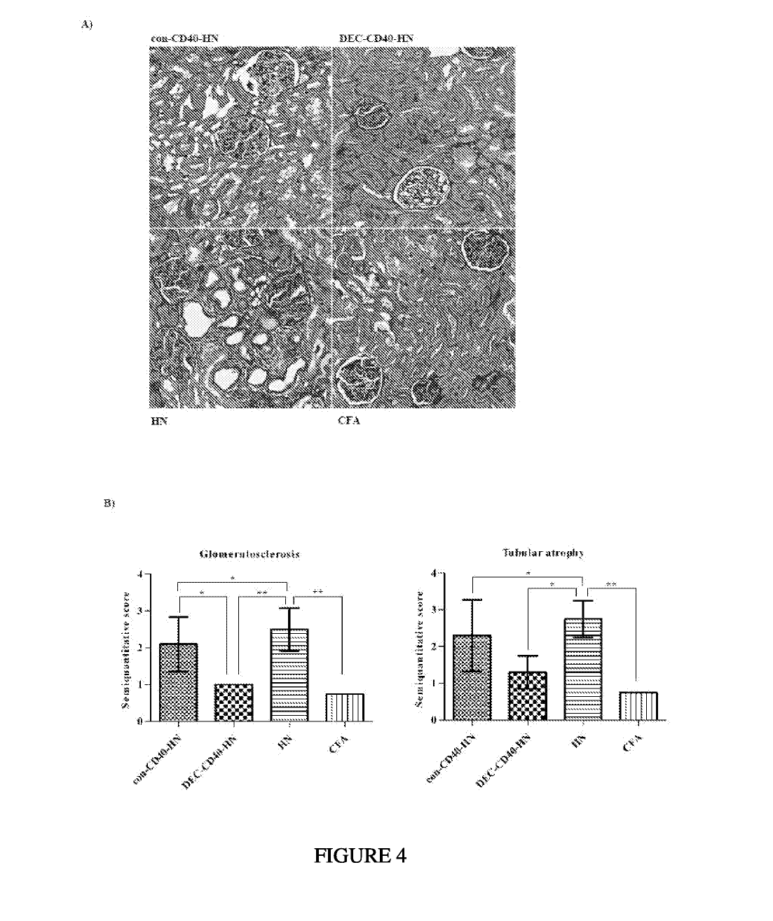

Renal structural injury was assessed histologically by evaluating glomerulosclerosis and tubular atrophy (FIG. 4A). Both con-CD40-HN and DECCD40-HN groups had significantly less glomerulosclerosis and tubular atrophy as compared to HN group (FIG. 4B).

2.5 scDEC-CD40 DNA Vaccination Reduces Immune Cell Infiltration and IgG Deposition

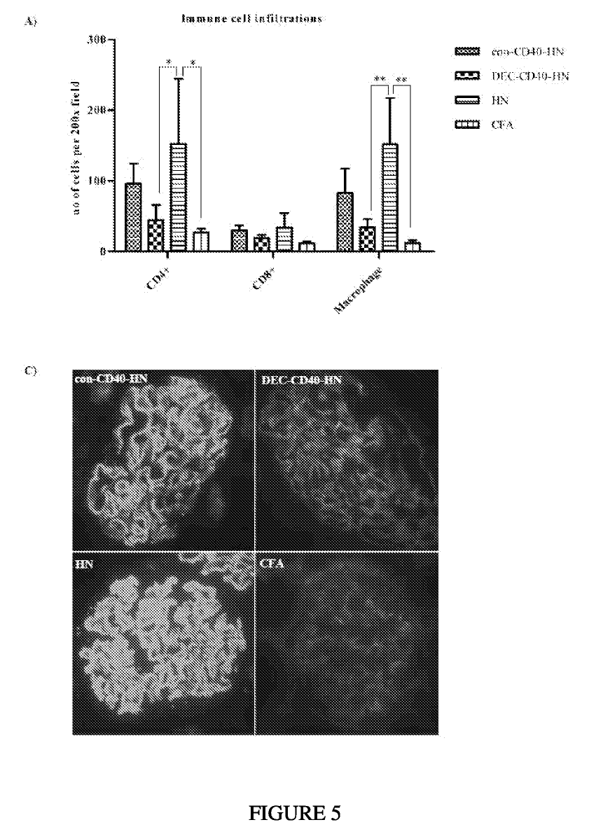

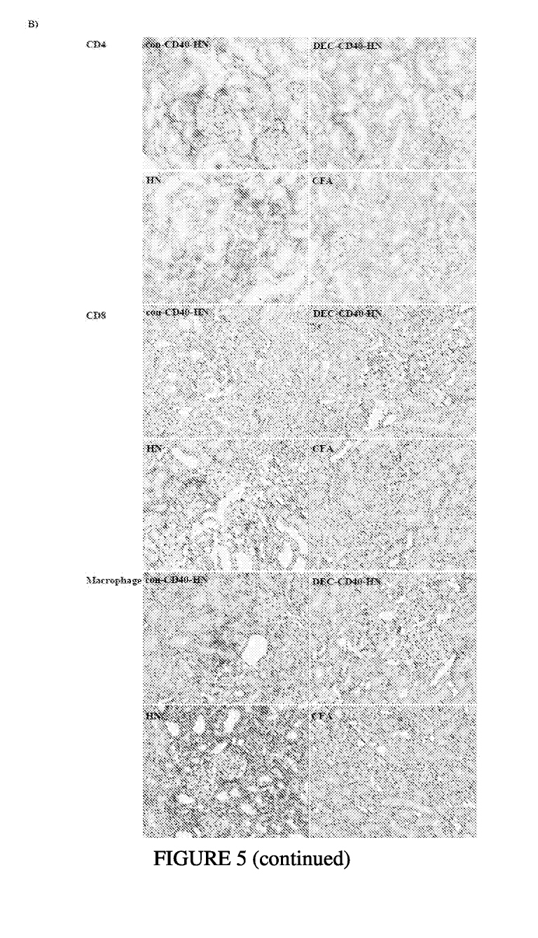

Immune cell infiltration was assessed by immunohistochemical staining of kidney sections with antibodies specific for CD4, CD8 and macrophages at week 12 post Fx1A/CFA immunization. As shown in FIG. 5A, the DEC-CD40-HN group had significantly less CD4⁺ and macrophage cell infiltrates than HN group, whereas con-CD40-HN group was not significantly different to the HN group. CD8⁺ cell infiltration did not differ significantly among the four groups. FIG. 5B shows representative histology from each group. Furthermore, glomerular IgG deposition (one of the major characteristics of HN) in the DEC-CD40-HN group was less dense than in other HN groups. Rats from the DEC-CD40-HN group had reduced deposition of IgG along the GBM whereas IgG deposition in HN and con-CD40-HN group showed dense linear deposition (FIG. 5C).

2.6 scDEC-CD40 DNA Vaccination Up-Regulates CTLA-4 mRNA Expression of Draining Lymph Node (DLN)

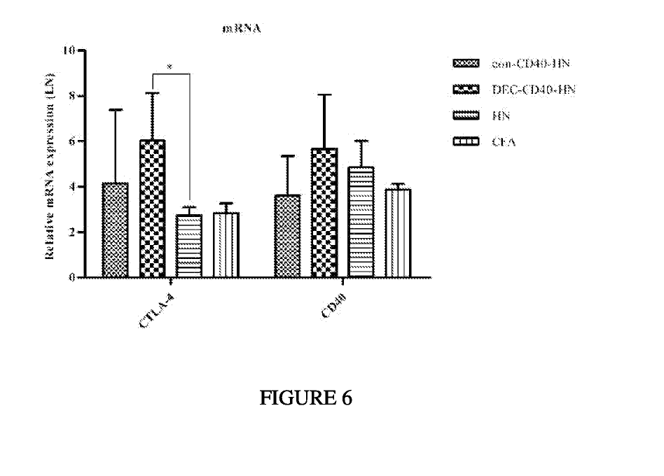

To determine the mechanism underlying the protective effects of CD40 DNA vaccination, expression was examined of several cytokines and co-stimulatory molecules in draining lymph nodes at week 12 post Fx1A/CFA immunization by semi-quantitative real-time PCR. CTLA-4 mRNA level was significantly up-regulated in the DEC-CD40-HN group, whereas CD40 mRNA remained unchanged (FIG. 6). No significant changes were detected for other cytokines and co-stimulatory molecules (data not shown).

### 2.7 In Vitro Effects of Serum Antibody on B Cell Activation and T Cell Proliferation

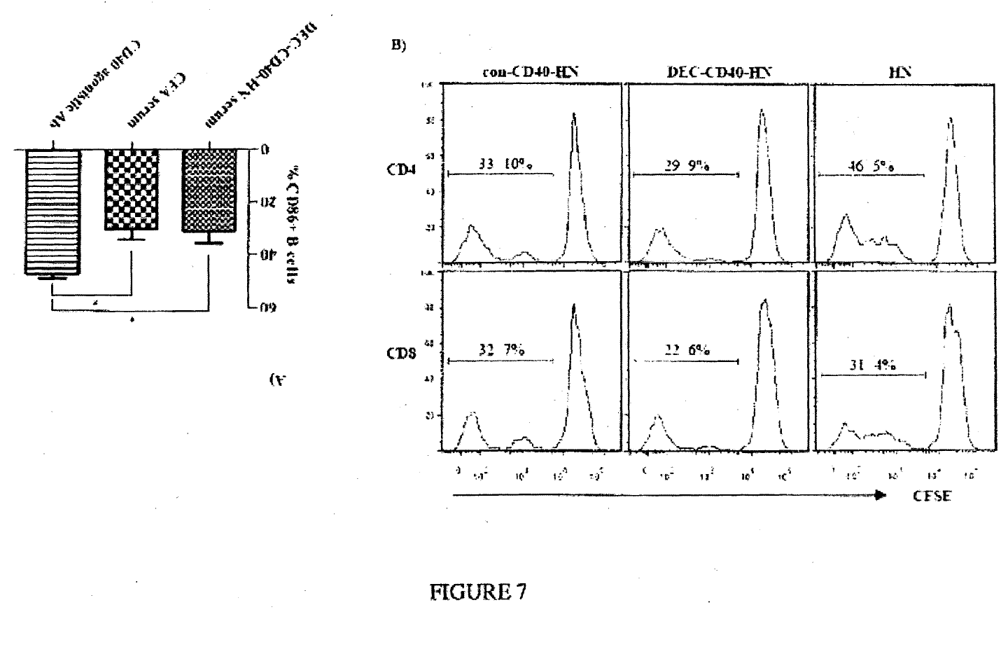

To determine whether the protective effects of DEC-CD40 vaccination on HN are due to an inhibitory or agonist CD40 antibody, the in vitro effects of serum on spleen cells from normal Lewis rat were assessed. Sera from DEC-CD40 or CFA groups collected at week 4 post Fx1A/CFA immunization were used with an agonist monoclonal antibody against CD40 as a positive control. B cell activation was determined by CD86 expression. As compared to agonist CD40 Ab, CD86 was not induced by serum from DEC-CD40 and CFA rats, consistent with a non-agonist antibody (FIG. 7A).

To determine whether anti-CD40 autoantibody, generated as a result of CD40 vaccination, can act as a blocking antibody functionally to inhibit T cell proliferation, the in vitro effects of serum from vaccinated rats on a mixed lymphocyte reaction (MLR) were assessed. CFSE-labelled responder cells (Lewis rat lymphocytes depleted of antigen-presenting cells (APC)) were co-cultured with stimulator cells (irradiated Wistar rat lymphocytes) in the presence or absence of 5% serum. A reduction in both CD4⁺ and CD8⁺ T cell proliferation with DEC-CD40-HN serum as compared to HN serum was observed (FIG. 7B).

2.8 In Vivo Assessment of B Cell Depletion after CD40 Vaccination

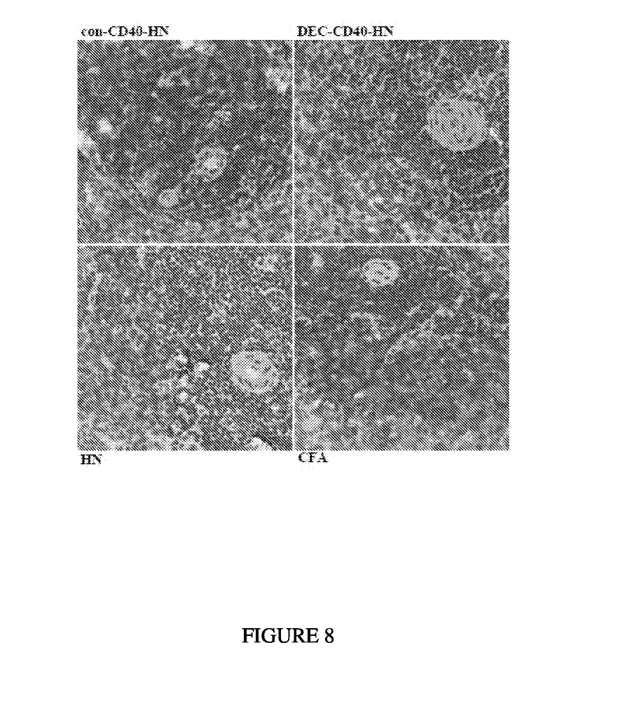

B cell numbers in spleens of vaccinated rats were assessed, as shown in FIG. 8. B cell numbers were not altered by CD40 vaccination, indicating that the antibodies induced by vaccination were not depleting.

### Example 3

### Prevention of an Autoimmune Response Against a Graft

DNA vaccines substantially as described in Example 1 are prepared and purified for administration. Lewis rats are injected intraperitoneally with a single injection of streptozotocin (60 mg/kg) to induce diabetes. Rats are then injected intramuscularly into rats followed by electroporation, which is known to improve uptake of the plasmid. In some cases adjuvants are used to improve immunogenicity of DC targeted vaccines.

Rat islets isolated from the pancreas of MHC mismatched Wistar rats are transplanted into diabetic rats under the renal capsule, essentially as described in Hu et al., Cell Transplantation (accepted for publication). Tolerance is then determined as described below at several time points out to about 100 days (long-term tolerance).

Islet transplants are examined for signs of rejection by testing blood sugar levels (BSL) on a daily basis. Allograft rejection is defined as BSL>16 mmol/L. Nephrectomies (removal of kidneys) are performed to confirm islet function following long term allograft survival.

Islet transplant function are confirmed immunohistochemically by the presence of insulin, substantially as described in Hu et al., supra and Wang et al., Journal of the American Society of Nephrology, 17: 697-706, 2006. Islet transplants are examined histologically for infiltration of immune cells including immunohistochemistry for regulatory T cells. B cell staining of the spleen is performed to exclude the potential for B cell depletion by antibodies.

Western blots and ELISAs are performed substantially as described in Wang et al., supra and Hu et al., supra, to determine the presence of CD40 serum antibodies in the transplant model.

Cell mediated and humoral immune responses are examined following DNA vaccination using flow cytometry for antibodies, B and T cell markers and cell markers for activation and proliferation substantially as described in Zeng et al., Journal of the American Society of Nephrology, 17, 465-474, 2006, and Wang et al., Journal of the American Society of Nephrology (accepted for publication).

Expression of cytokines and immune molecules, including regulatory molecules are investigated using real time PCR following DNA vaccination substantially as described in Wang et al., supra.

Proliferation assays and Elispot analysis for specific cytokines are performed examined to determine the effect of DNA vaccination on the alloreactive T cell and Treg cell responses in vitro substantially as described in Watson et al., Current Opinion in Organ Transplantation, 14, 357-363, 2004; and Wang et al, supra.

1. A recombinant nucleic acid encoding a polypeptide comprising the following linked regions:
(i) a region that binds to a cell surface protein expressed on a dendritic cell; and
(ii) a co-stimulatory molecule or an immunogenic fragment thereof.

2. The recombinant nucleic acid of claim 1, wherein the region that binds to a cell surface protein expressed on a dendritic cell comprises a variable region of an antibody that binds to the cell surface protein.

3. The recombinant nucleic acid of claim 2, wherein the region that binds to a cell surface protein expressed on a dendritic cell comprises a Fv of an antibody that binds to the cell surface protein.

4. The recombinant nucleic acid of any one of claims 1 to 3, wherein the cell surface protein expression on a dendritic cell is selected from the group consisting of a mannose receptor, chemokine receptor CCR1, CD80, CD86, CD11c, DEC-205, a Toll-like receptor (TLR), a C-type lectin and a Fcγ receptor (FcγR).

5. The recombinant nucleic acid of any one of claims 1 to 3, wherein the cell surface protein expression on a dendritic cell is an endocytic receptor or a C-type lectin.

6. The recombinant nucleic acid of claim 1, wherein the cell surface protein expression on a dendritic cell is DEC-205, CLEC9A or DC-SIGN.

7. The recombinant nucleic acid of any one of claims 1 to 5, wherein the co-stimulatory molecule expressed by an antigen presenting cell.

8. The recombinant nucleic acid of claim 7, wherein the co-stimulatory molecule is a co-stimulatory molecule expressed by a B cell.

9. The recombinant nucleic acid of claim 8, wherein the co-stimulatory molecule is selected from the group consisting of CD40, CD80, CD84, CD86, CD150, CD229, B7-H4, programmed death 1 (PD-1), transmembrane activator and CAML-interactor (TACI), B cell-activating factor receptor (BAFF R), B cell maturation protein (BCMA), CD30 ligand, OX40 ligand and NTB-A.

10. The nucleic acid of claim 7, wherein the co-stimulatory molecule is CD40.

11. The nucleic acid according to any one of claims 1 to 10, additionally comprising a region encoding a T helper epitope.

12. The nucleic acid of claim 11, wherein the T helper epitope is the P30 epitope of tetanus toxoid (SEQ ID NO: 5).

13. A recombinant nucleic acid encoding a polypeptide comprising the following linked regions (listed in amino to carboxy order):
(i) an antibody variable region that binds specifically to DEC-205, DC-SIGN or CLEC9A; and
(ii) CD40 or an immunogenic fragment thereof.

14. A recombinant nucleic acid encoding a polypeptide comprising the following linked regions (listed in amino to carboxy order):
(i) an antibody variable region that binds specifically to DEC-205, DC-SIGN or CLEC9A;
(ii) CD40 or an immunogenic fragment thereof; and
(iii) P30 epitope of tetanus toxoid (SEQ ID NO: 5)

15. The recombinant nucleic acid of any one of claims 1 to 14, operably linked to a promoter operable in a mammalian cell.

16. A recombinant polypeptide encoded by the recombinant nucleic acid of any one of claims 1 to 15.

17. An immunogenic composition comprising the recombinant nucleic acid of any one of claims 1 to 15 or the recombinant polypeptide of claim 16 and a pharmaceutically acceptable carrier.

18. A DNA vaccine comprising the recombinant nucleic acid of any one of claims 1 to 15.

19. A vaccine comprising the recombinant nucleic acid of any one of claims 1 to 15 or the recombinant polypeptide of claim 16.

20. A method of treating or preventing an autoimmune disease, an inflammatory disease or allergy in a subject, the method comprising immunizing the subject with the recombinant nucleic acid of any one of claims 1 to 15 or the recombinant polypeptide of claim 16 or the immunogenic composition of claim 17 or the DNA vaccine of claim 18 or the vaccine of claim 19.

21. The method of claim 20, wherein the autoimmune disease is autoimmune nephritis.

22. A method for inducing an immune response against an immune cell of a subject, the method comprising immunizing the subject with the recombinant nucleic acid of any one of claims 1 to 15 or the recombinant polypeptide of claim 16 or the immunogenic composition of claim 17 or the DNA vaccine of claim 18 or the vaccine of claim 19, wherein the immune response neutralizes activity of the co-stimulatory molecule without depleting cells expressing the co-stimulatory molecule.

23. A method for preventing or suppressing an immune response in a subject, the method comprising immunizing the subject with the recombinant nucleic acid of any one of claims 1 to 15 or the recombinant polypeptide of claim 16 or the immunogenic composition of claim 17 or the DNA vaccine of claim 18 or the vaccine of claim 19, wherein the immune response neutralizes activity of the co-stimulatory molecule without depleting cells expressing the co-stimulatory molecule.

24. A method of transplanting a cell, tissue or organ into a subject, the method comprising:
(i) performing the method of claim 23 to thereby suppress and immune response in the subject; and
(ii) transplanting the cell, tissue or organ into the subject.

25. The method of claim 24, wherein the tissue or organ is a pancreas, a pancreatic islet, pancreatic β cells, a kidney or a liver or part thereof.
